(19) United States
(12) Patent Application Publication
(10) Pub. No.: US 2023/0310317 A1
(43) Pub. Date: Oct. 5, 2023

(54) SOL-GEL COMPOSITION

(71) Applicant: THE UNIVERSITY OF QUEENSLAND, St. Lucia, Queensland (AU)

(72) Inventors: Harendra PAREKH, Chapel Hill, Queensland (AU); Preeti PANDEY, Pallara, Queensland (AU)

(21) Appl. No.: 18/023,274

(22) PCT Filed: Aug. 27, 2021
(86) PCT No.: PCT/AU2021/050991

§ 371 Date: Feb. 24, 2023

(30) Foreign Application Priority Data
Aug. 27, 2020 (AU) 2020903065

(51) Int. Cl. A61K 9/06; B01J 13/12; B01J 13/00; A61K 47/32; A61K 31/192; A61K 9/19; A61K 47/26; A61K 47/10; A61K 31/58; A61K 31/12; A61K 31/337; A61K 36/185; A61K 31/355; A61K 31/593; A61K 38/13; A61K 47/36

(52) U.S. Cl. CPC A61K 9/06; B01J 13/125; B01J 13/0065; A61K 47/32; A61K 31/192; A61K 9/19; A61K 47/26; A61K 47/10; A61K 31/58; A61K 31/12; A61K 31/337; A61K 36/185; A61K 31/355; A61K 31/593; A61K 38/13; A61K 47/36; A61K 2236/333

(57) ABSTRACT

This invention relates to, in some aspects, a method of preparing a gel-based composition or a thermo-responsive sol-gel composition, including the steps of: (a) providing a mixture of a first aqueous solution comprising a first poloxamer and/or a first poloxamine with a solvent solution comprising a water miscible solvent and a hydrophobic therapeutic agent, wherein the water miscible solvent has a boiling point of less than 105° C. at atmospheric pressure and wherein the first aqueous solution and/or the solvent solution further comprise a surfactant; (b) substantially removing the water miscible solvent and water from the mixture in (a) to produce a micelle composition; and (c) contacting the micelle composition with a second aqueous solution comprising a second poloxamer and/or a second poloxamine to thereby prepare the thermo-responsive sol-gel composition. In other aspects, the present invention relates to a method of preparing a micelle composition, a thermo-responsive sol-gel composition or a gel-based composition for therapeutic use, and to methods of using the compositions.

### FIELD OF THE INVENTION

This invention relates to a sol-gel composition. In particular, the invention relates to a sol-gel composition including a carrier micelle containing a hydrophobic, aqueous insoluble therapeutic agent therein and methods of making and using same.

### BACKGROUND TO THE INVENTION

The nasal airway passages play a crucial role in upper airway homeostasis with an abundance of airborne pathogens being drawn into the sinuses with each breath, resulting in a high probability of localised infection, chronic inflammation and/or allergic responses in susceptible patient groups (Parikh et al., 2014). Sinusitis, rhinitis or rhinosinusitis is an inflammatory condition of the nasal and paranasal sinus mucosa resulting in nasal discharge/congestion, nasal blockage, facial pain/pressure and reduction of sense of smell (Rosenfeld et al., 2007; Fokkens et al., 2007). Chronic rhinosinusitis (CRS), defined by the persistence of symptoms beyond 3 months, is among the commonest chronic medical complaints cited in the United States, affecting nearly 16% of the general population, with the increasing incidence and prevalence accounting for 13 million physician visits annually costing an estimated US$6 billion/year (Blackwell et al., 2014; Piromchai et al., 2013). Similarly, allergic rhinitis (AR), which is typically triggered by environmental allergens such as pollen, pet hair, dust, or mold, is defined by symptoms of sneezing, nasal pruritus, airflow obstruction, and mostly clear nasal discharge caused by IgE-mediated reactions against such allergens. The prevalence of allergic rhinitis in Western countries may be as high as 30% (Wheatley and Togias, 2015).

Treatment of CRS typically requires medical and/or surgical intervention, with the former often involving a combination of antibiotics, nasal decongestants, topical nasal/oral steroids as well as saline irrigation. Similarly, treatment of AR generally involves pharmacotherapy, such as antihistamines, intranasal steroids, and leukotriene-receptor antagonists, and/or immunotherapy. The vast majority of these therapeutic agents, however, invariably lack sufficient residence time and physical integrity, limiting adherence to mucosal tissue.

With respect to CRS and AR, the efficacy of intranasal steroid sprays, drops and other conventional delivery methods are also compromised by poor drug delivery and retention, which is not helped by underlying postoperative oedema, crusting and secretions that ultimately lead to poor patient compliance (Rizan and Elhassan, 2016). Medical therapy remains the foundation of long-term care of chronic rhinosinusitis, particularly in surgically recalcitrant cases, although effective drug delivery remains a major obstacle to achieving this (Liang and Lane, 2013).

Accordingly, there remains a need for formulations of therapeutic agents, particularly those hydrophobic in nature, that reduce anterior and posterior leakage, provide protection to the drug from enzymatic degradation, increase the rate of drug dissolution, improve residence time of the formulation with the nasal mucosa while enhancing drug uptake across the epithelium.

### SUMMARY OF THE INVENTION

The present invention is directed to a sol-gel composition including a hydrophobic therapeutic agent-containing micelle and methods of preparing and using the same.

In a first aspect, the invention provides a method of preparing a thermo-responsive sol-gel composition, including the steps of:
(a) providing a mixture of a first aqueous solution comprising a first poloxamer and/or a first poloxamine with a solvent solution comprising a water miscible solvent and a hydrophobic therapeutic agent, wherein the water miscible solvent has a boiling point of less than 105° C. at atmospheric pressure and wherein the first aqueous solution and/or the solvent solution further comprise a surfactant;
(b) substantially removing the water miscible solvent and water from the mixture in (a) to produce a micelle composition;
(c) contacting the micelle composition with a second aqueous solution comprising a second poloxamer and/or a second poloxamine to thereby prepare the thermo-responsive sol-gel composition.

In a second aspect, the invention provides a method of preparing a gel-based composition, including the steps of:
(a) providing a mixture of a first aqueous solution comprising a first poloxamer and/or a first poloxamine with a solvent solution comprising a water miscible solvent and a hydrophobic therapeutic agent, wherein the water miscible solvent has a boiling point of less than 105° C. at atmospheric pressure and wherein the first aqueous solution and/or the solvent solution further comprise a surfactant;
(b) substantially removing the water miscible solvent and water from the mixture in (a) to produce a micelle composition;
(c) contacting the micelle composition with a second aqueous solution comprising a second poloxamer, a second poloxamine and/or a further polymer to thereby prepare the gel-based composition.

In a third aspect, the invention resides in a method of preparing a micelle composition, including the steps of:
(a) providing a mixture of a first aqueous solution comprising a first poloxamer and/or a first poloxamine with a solvent solution comprising a water miscible solvent and a hydrophobic therapeutic agent, wherein the water miscible solvent has a boiling point of less than 105° C. at atmospheric pressure and wherein the first aqueous solution and/or the solvent solution further comprise a surfactant;
(b) substantially removing the water miscible solvent and water from the mixture in (a) to thereby produce the micelle composition.

For the first, second and third aspects, the method may further include the steps of:
(a) mixing the first aqueous solution and the solvent solution; and/or
(b) preparing the first aqueous solvent, the second aqueous solvent and/or the solvent solution.

In one embodiment of the above aspects, the first aqueous solution comprises the surfactant.

Suitably for the method of the first, second and third aspects, the water miscible solvent is or comprises a ketone and/or an alcohol (especially a primary alcohol). More particularly, the water miscible solvent can be selected from the group consisting of acetone, methanol, ethanol, propanol, butanol, pentanol, hexanol and any combination thereof.

In one embodiment of the above aspects, the first and/or the second poloxamer are or comprise P407.

In particular embodiments of the method of the first, second and third aspects, the step of removing the water is performed at least in part by lyophilization.

In some embodiments of the aforementioned aspects, the step of removing the water miscible solvent is performed at reduced pressure, for example at least in part by rotary evaporation or using a rotary evaporator.

Referring to the above aspects, the step of removing the water miscible solvent is suitably performed at a temperature from about 25° C. to about 35° C. More particularly, the step of removing the water miscible solvent can be performed at a temperature from about 32° C. to about 34° C.

In a fourth aspect, the invention provides a thermo-responsive sol-gel composition or a gel-based composition for therapeutic use comprising:
an aqueous solution;
a carrier micelle disposed within the aqueous solution and including a poloxamer and/or a poloxamine and a surfactant and having a hydrophobic core; and
a hydrophobic therapeutic agent disposed within the hydrophobic core of said carrier micelle.

With respect to the first and fourth aspects, the thermo-responsive sol-gel composition suitably has a visual gelation temperature below about 35° C. More particularly, the gelation temperature is between about 20° C. to about 32° C.

In particular embodiments of the first and fourth aspects, the thermo-responsive sol-gel composition has a viscosity of:
(a) less than about 0.15 Pa·s at about 22° C.; and
(b) greater than about 0.3 Pa·s at about 30° C. or about 35° C., especially greater than about 0.4 Pa·s at about 30° C. or about 35° C.

Again, referring to the first and fourth aspects, the thermo-responsive sol-gel composition suitably has a gel strength of greater than about 500 Pa at about 30° C. or at about 35° C. or more particularly greater than about 1000 Pa at about 30° C. or at about 35° C.

Suitably, for the aforementioned aspects, the surfactant is selected from the group consisting of a polyoxyethylated sorbitan fatty ester, a polyoxyethylated glycol monoether, a polyoxyethylated glyceride, n-dodecyl tetra (ethylene oxide), a polyoxyethylated fatty acid, a polyoxyethylated castor oil, a sucrose ester, a lauroyl macroglyceride, a polyglycolyzed glyceride and any combination thereof. More particularly, the surfactant suitably is or comprises polyoxyethylene sorbitan monooleate.

In particular embodiments of the above aspects, the hydrophobic therapeutic agent is selected from the group consisting of a steroid, an anticancer agent, an antifungal agent, an anti-inflammatory agent, a sex hormone, an immunosuppressant, an antiviral agent, an antibacterial agent, an anti-fibrotic agent, an antihistamine agent, a vitamin, a plant extract and any combination thereof.

In one embodiment of the present aspect, the carrier micelle is about 10 nm to about 25 nm in diameter.

In particular embodiments of the present aspect, the poloxamer is or comprises P407.

In a fifth aspect, the invention resides in a thermo-responsive sol-gel composition prepared by the method of the first aspect.

In a sixth aspect, the invention relates to a gel-based composition prepared by the method of the second aspect.

In a seventh aspect, the invention provides a micelle composition prepared by the method of the third aspect.

In an eighth aspect, the invention resides in a method of administering a hydrophobic therapeutic agent to a subject, the method including the step of administering the thermo-responsive sol-gel composition of the fourth or fifth aspects, the gel-based composition of the fourth or sixth aspects and/or the micelle composition of the seventh aspect to the subject.

In a ninth aspect, the invention provides a method of preventing and/or treating a disease, disorder or condition in a subject, including the step of administering to the subject a therapeutically effective amount of the thermo-responsive sol-gel composition of the fourth or fifth aspects, the gel-based composition of the fourth or sixth aspects and/or the micelle composition of the seventh aspect to thereby prevent and/or treat the disease, disorder or condition.

In a tenth aspect, the invention provides a thermo-responsive sol-gel composition, a gel-based composition or a micelle composition, according to the fourth, fifth, sixth or seventh aspects, for use in preventing and/or treating a disease, disorder or condition in a subject.

In an eleventh aspect, the invention resides in a use of the thermo-responsive sol-gel composition of the fourth or fifth aspects, the gel-based composition of the fourth or sixth aspects and/or the micelle composition of the seventh aspect, in the manufacture of a medicament for preventing and/or treating a disease, disorder or condition in a subject.

Referring to the invention of the ninth, tenth or eleventh aspects, the disease, disorder or condition suitably is or comprises a respiratory disease, disorder or condition.

As used herein, except where the context requires otherwise, the term “comprise” and variations of the term, such as “comprising”, “comprises” and “comprised”, are not intended to exclude further elements, components, integers or steps but may include one or more unstated further elements, components, integers or steps.

It will be appreciated that the indefinite articles “a” and “an” are not to be read as singular indefinite articles or as otherwise excluding more than one or more than a single subject to which the indefinite article refers. For example, “a” surfactant includes one surfactant, one or more surfactants and a plurality of surfactants.

### BRIEF DESCRIPTION OF THE FIGURES

FIG. 1. Method 1 resulted in a highly turbid MF-loaded sol-gel formulation.

FIG. 2. Method 2 resulted in an opaque MF-loaded sol-gel formulation.

FIG. 3. Method 3 resulted in a translucent MF-loaded sol-gel formulation.

FIG. 4. Method 4 resulted in a near-transparent MF-loaded sol-gel formulation, confirmed via turbidimetric measurement (see Table 3).

FIG. 5. Sol-gels containing blank micelles (left) and 0.1% w/w MF (right) prepared by ‘Method 4A’ after 24 hr storage at 2-8° C.

FIG. 6. Rheogram profile of MF sol-gels prepared by (A) Method 1; (B) Method 2; (C) Method 3; (D) Method 4; (E) Method 4A.

FIG. 7. Sol-gels containing blank micelles (top left), 0.1% w/w vitamin D (top right) and rheogram of gelation profile (bottom).

FIG. 8. Sol-gels containing blank micelles (top left), 0.1% w/w vitamin E (top right) and rheogram of gelation profile (bottom).

FIG. 9. Sol-gels containing blank micelles (top left), 0.1% w/w cyclosporine (top right) and rheogram of gelation profile (bottom).

FIG. 10. A: Sol-gel formulations containing MF-micelles (left) with surfactant (0.8 g (8% w/w) Tween® 80) and (right) without surfactant (both formulations containing a total of 14.5% w/w P407; 1.1 g in Phase 1 and 0.35 g in Phase 2) after cold storage for 24 h and appearance after standing at room temperature (22-24° C.) for 60 minutes. B: Sol-gel formulations containing MF-micelles (left) with surfactant (0.8 g (8% w/w) Tween® 80) and (right) without surfactant (both formulations containing a total of 14.5% w/w P407; 1.1 g in Phase 1 and 0.35 g in Phase 2) and appearance after standing at room temperature (22-24° C.) for >48 h.

FIG. 11. Gel formulations containing KP-loaded micelles and (A) a carbomer based gel or (B) a poloxmer based gel, both at room temperature.

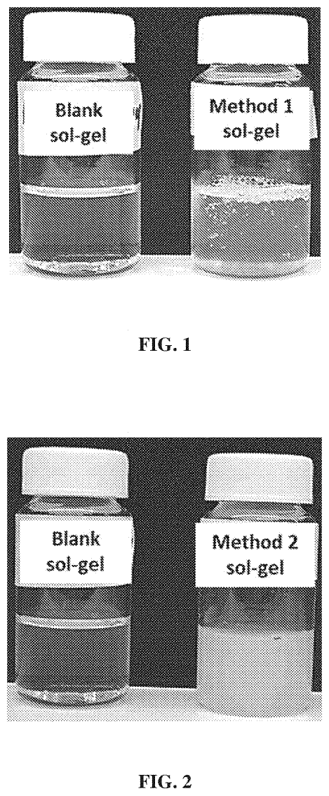

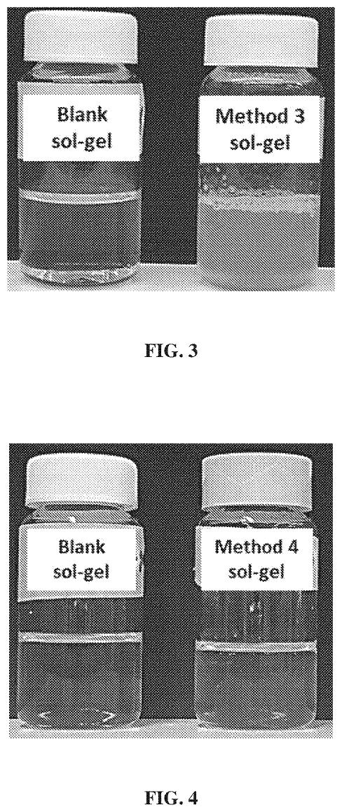

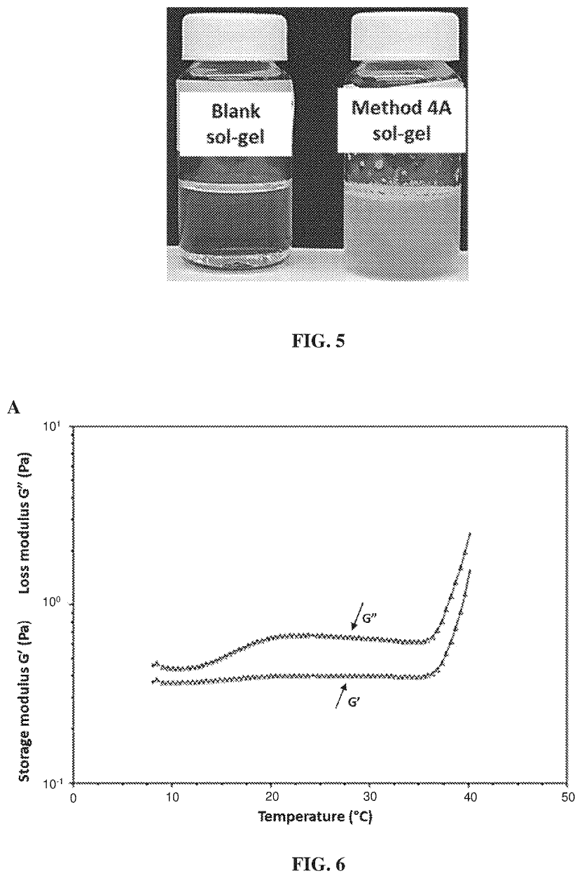

### DETAILED DESCRIPTION OF THE INVENTION

The present invention is predicated, at least in part, on the surprising discovery of a method for producing a specifically designed sol-gel composition having a hydrophobic therapeutic agent contained within a micellar component that maintains the therapeutic agent in a soluble state and having a gelation or transition temperature, which allows the composition to adhere to a mucosal surface upon administration thereto. Possible advantages of this sol-gel composition include enhanced drug absorption and residence time at the target site, such as a mucosal surface (e.g., the nasal mucosa) and the skin, and thereby allowing for reduced dosages and dosing frequencies, reduced irritation at the site of application, improved patient compliance, combined local and systemic drug delivery and the avoidance of anterior leakage and post-nasal dripping of drug for nasal applications.

Accordingly, in one aspect, the invention provides a method of preparing a thermo-responsive sol-gel composition, including the steps of:
(a) providing a mixture of a first aqueous solution comprising a first poloxamer and/or a first poloxamine with a solvent solution comprising a water miscible solvent and a hydrophobic therapeutic agent, wherein the water miscible solvent has a boiling point of less than 105° C. at atmospheric pressure and wherein the first aqueous solution and/or the solvent solution further comprise a surfactant;
(b) substantially removing the water miscible solvent and water from the mixture in (a) to produce a micelle composition;
(c) contacting or mixing the micelle composition with a second aqueous solution comprising a second poloxamer and/or a second poloxamine to thereby prepare a thermo-responsive sol-gel composition.

In a related aspect, the invention provides a method of preparing a gel-based composition, including the steps of:
(a) providing a mixture of a first aqueous solution comprising a first poloxamer and/or a first poloxamine with a solvent solution comprising a water miscible solvent and a hydrophobic therapeutic agent, wherein the water miscible solvent has a boiling point of less than 105° C. at atmospheric pressure and wherein the first aqueous solution and/or the solvent solution further comprise a surfactant;
(b) substantially removing the water miscible solvent and water from the mixture in (a) to produce a micelle composition;
(c) contacting the micelle composition with a second aqueous solution comprising a second poloxamer, a further polymer and/or a second poloxamine to thereby prepare the gel-based composition.

In a further related aspect, the invention provides a method of preparing a micelle composition, including the steps of:
(a) providing a mixture of a first aqueous solution comprising a first poloxamer and/or a first poloxamine with a solvent solution comprising a water miscible solvent and a hydrophobic therapeutic agent, wherein the water miscible solvent has a boiling point of less than 105° C. at atmospheric pressure and wherein the first aqueous solution and/or the solvent solution further comprise a surfactant;
(b) substantially removing the water miscible solvent and water from the mixture in (a) to thereby produce the micelle composition.

In particular embodiments, the method of the aforementioned aspects further includes the initial step of mixing the first aqueous solution and the solvent solution.

Additionally, the method of the aforementioned aspects further includes the step of preparing the first and/or second aqueous solutions and/or the solvent solution.

The statements which follow apply equally to the aforementioned aspects.

It will be appreciated that the micelle composition may be formulated in a manner that is compatible with its end use. In particular embodiments, the micelle composition is or comprises a solid or semi-solid residue (e.g., a paste, a gel, a viscous liquid). In some embodiments, the micelle composition is or comprises a micelle powder.

The descriptor “gel” refers to the physical properties of the compositions according to the invention, which are generally semi-solid systems that include a liquid or liquid-like component and optionally solid particles and other components, such as carrier micelles, dispersed or disposed therein.

The term “thermo-responsive sol-gel”, as generally used herein, refers to a composition, which undergoes a phase transition from a solution or liquid phase to a gel phase (e.g., the conversion of a liquid or flowable form with a viscosity of about 0.05 Pascal-seconds or less to a gel or relatively semi-solid form with a viscosity of at least about 0.4 Pascal-seconds) or vice versa when the temperature is raised above or reduced below a critical value, which is referred to herein as a “gelation temperature” or “transition temperature”. Preferably the phase transition from a liquid to a gel and vice versa occurs in less than 10 minutes (e.g., 5 sec, 10 sec, 15 sec, 30 sec, 1 min, 2 min, 3 min, 4 min, 5 min, 6 min, 7 min, 8 min, 9 min, 10 min and any range therein), more particularly in less than 5 minutes and even more particularly in less than 2 minutes.

It will be understood that the present method suitably results in the formation of a plurality of carrier micelles within the gel-based composition or the sol-gel composition. As used herein, the term “micelle” refers to an aggregation of molecules wherein hydrophobic portions of these molecules comprise the interior of the aggregation (i.e., the hydrophobic polymeric core) and hydrophilic portions of the molecules comprise the exterior of the aggregation (i.e., the outer hydrophilic polymeric layer). In this regard, micelles are spontaneously formed by amphiphilic compounds in water above a critical solute concentration, the critical micellar concentration (CMC), and at solution temperatures above the critical micellar temperature (CMT). There are many ways to determine CMC, including surface tension measurements, solubilization of water insoluble dye, or a fluorescent probe, conductivity measurements, light scattering, and the like.

By “poloxamer” is meant a nonionic triblock copolymers composed of a central hydrophobic chain of polyoxypropylene (poly(propylene oxide) or PPO) flanked by two hydrophilic chains of polyoxyethylene (poly(ethylene oxide) or PEO). Such poloxamers may be linear or branched, and include notably tri-blocks or tetra-blocks copolymers. Exemplary poloxamers include F87, F88, F98, F108, F38, F127 (P407), L35, P84, P85, L62, L63, L64, P65, F68, L72, P75, F77, P105, L42, L43, L44, P103, P104, P105, L81, L101, L121, L122 and P123.

It will further be appreciated that the nomenclature of poloxamers relates to their monomeric composition. The first two digits of a poloxamer number, multiplied by 100, gives the approximate molecular weight of the hydrophobic polyoxypropylene block. The last digit, multiplied by 10, gives the approximate weight percent of the hydrophilic polyoxyethylene content. For example, poloxamer 407 (P407) describes a polymer containing a polyoxypropylene hydrophobe of about 4,000 g/mol with a hydrophilic polyoxyethylene block content of about 70% of the total molecular weight. Most preferred poloxamers are ones that are pharmaceutically acceptable for the intended route of administration of the gel-based composition or the sol-gel composition.

It will be appreciated that a first poloxamer and/or a first poloxamine that make up the carrier micelle may be any physiologically acceptable poloxamer or poloxamine known in the art that is capable of micelle formation. Additionally, it is envisaged that the first poloxamer and/or the first poloxamine may include a plurality (e.g., 2, 3, 4, 5 etc or more) of poloxamers and poloxamines respectively.

In particular embodiments, the first poloxamer is selected from the group consisting of P407, P124, P188, P237, P338 and any combination thereof. More particularly, the first poloxamer suitably is or comprises P407 (also known as F127).

The term “poloxamine” denotes a polyalkoxylated symmetrical block copolymer of ethylene diamine conforming to the general type [(PEG)ₓ-(PPG)_{y}]₂-NCH₂CH₂N-[(PPG)_{γ}-(PEG)_{χ}]₂. Each poloxamine name is followed by an arbitrary code number, which is related to the average numerical values of the respective monomer units denoted by X and Y. Poloxamines are typically prepared from an ethylene diamine initiator and synthesized using the same sequential order of addition of alkylene oxides as used to synthesize poloxamers. Structurally, the poloxamines generally include four alkylene oxide chains and two tertiary nitrogen atoms, at least one of which is capable of forming a quaternary salt. Poloxamines are usually also terminated by primary hydroxyl groups.

Poloxamines are commercially available in a wide range of EO/PO (ethyleneoxide (EO)/propylene oxide (PO)) ratios and molecular weights under the tradename Tetronic® (BASF). Exemplary poloxamines include T304, T701, T707, T901, T904, T908, T1107, T1301, T1304, T1307, T90R4, T150R1 and T1508, whose properties are shown in table 1.

TABLE 1
Properties of various poloxamines

| | Mw | EO units | PO units per | |
|---|---|---|---|---|
| Tetronic | (Da) | per block (a) | block (b) | HLB |
| 304 | 1650 | 3.7 | 4.3 | 12-18 |
| 701 | 3600 | 2.1 | 14.0 | 1-7 |
| 901 | 4700 | 2.7 | 18.2 | 1-7 |
| 904 | 6700 | 15 | 17 | 12-18 |
| 908 | 25000 | 114 | 21 | >24 |
| 1107 | 15000 | 60 | 20 | 18-23 |
| 1301 | 6800 | 4 | 26 | 1-7 |
| 1304 | 10500 | 21.4 | 27.1 | 12-18 |
| 1307 | 18000 | 72 | 23 | >24 |
| 90R4 | 6900 | 16 | 18 | 1-7 |
| 150R1 | 8000 | 5 | 30 | 1-7 |

As used herein, the term “block copolymer” can refer to a polymer in which adjacent polymer segments or blocks are different, i.e., each block comprises a unit derived from a different characteristic species of monomer or has a different composition of units.

The further polymer may be any as are known in the art suitable for use in a gel-based composition. In this regard, the further polymer may or may not demonstrate or possess a sol-gel transition property. Exemplary further polymers are outlined below, and these may be used individually or in combination as required.

Suitable examples of further polymers include natural polymers such as: (a) proteins like gelatin, casein, collagen, egg whites; and (b) polysaccharides like guar gum, karaya gum, acacia or gum arabic, tragacanth, bug bean gum, pectin, starch, xanthan gum, dextran, succinoglucon.

The further polymer may also include semisynthetic polymers, such as cellulose subordinates like carboxymethyl cellulose, ethylcellulose, hydroxyethyl cellulose, hydroxypropyl cellulose, hydroxypropylmethyl cellulose, magnesium aluminium silicate (Veegum®), methylcellulose and sodium alginate.

Additional examples of suitable further polymers for use in the present invention include synthetic polymers, such as carbomers (Carbopol® 910, Carbopol® 934, Carbopol® 940, Carbopol® 941).

The term “carbomer” is intended to denote an acrylic acid polymer cross-linked with a polyfunctional compound, such as sugar polyalkenyl ethers (e.g., pentaerythritol allyl ethers, allyl disaccharide ethers). Examples of suitable carbomers include carbomer 910, carbomer 934, carbomer 934P, carbomer 940, carbomer 941, carbomer 971P, carbomer 974P, carbomer 980 or carbomer 981. The term “carbomer” may also include a chain alkyl emethacrylate acrylic acid copolymer long crosslinked with depentaerythritol allyl ethers, for example carbomer 1342, Carbopol® 1382, Carbopol®2984 or Carbopol® 5984.

In particular embodiments, the first and/or second poloxamer and/or poloxamine have a molecular weight of between about 1,000 to about 20,000 (e.g., about 1000, 1500, 2000, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 14000, 14500, 15000, 15500, 16000, 16500, 17000, 17500, 18000, 18500, 19000, 19500, 20000 and any range therein).

In certain embodiments, the first and/or second poloxamer and/or poloxamine have a ratio EO units per block to PO units per block of between about 6:1 to about 1:6 (e.g., about 6:1, 5.5:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5, 1:5.5, 1:6 and any range therein).

Further, it will be understood that the second poloxamer, further polymer and/or a second poloxamine may be any physiologically acceptable poloxamer, polymer and/or poloxamine (inclusive of combinations thereof) known in the art that preferably includes a sol-gel transition property. For the gel-based composition, however, it will be appreciated that the second poloxamer, further polymer and/or a second poloxamine need not necessarily include a sol-gel transition property. The term “a sol-gel transition property” means a property of said poloxamer and/or poloxamine in which a sol phase (i.e., solution phase or liquid phase) is converted to a gel phase (i.e., a sol-gel phase transition) by one or more specific stimuli. The specific stimuli may vary according to the kind of polymer, and may, for example, include a change in temperature, a change in pressure, a change in pH, or addition of salts, and the like, but the present invention is not limited thereto.

Suitably, the second poloxamer, further polymer and/or the second poloxamine may be any as are known in the art, such as those hereinbefore described. In one embodiment, the second poloxamer is or comprises P407 (F127). To this end, it will be appreciated that the first poloxamer and/or poloxamine of the carrier micelle may be the same or identical to the second poloxamer and/or poloxamine such as for the purposes of compatibility of the carrier micelle within the second aqueous solution.

For the first aqueous solution, the first poloxamer and/or poloxamine may be present in in an amount from about 0.5% to about 30% or any range therein such as, but not limited to, about 5% to about 25%, or about 10% to about 20% by weight of the first aqueous solution. In particular embodiments of the present invention, the first poloxamer and/or poloxamine is present in an amount of about 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or any range therein, by weight of the first aqueous solution. It will be apparent to the skilled artisan that the amount of the first poloxamer and/or poloxamine present in the first aqueous solution may be determined, at least in part, by the CMC and/or CMT of the first poloxamer and/or poloxamine.

For the carrier micelle, the first poloxamer and/or poloxamine may be present in an amount from about 20% to about 99.5% or any range therein such as, but not limited to, about 30% to about 95%, or about 40% to about 90% by weight of the carrier micelle. In particular embodiments of the present invention, the first poloxamer and/or poloxamine is present in an amount of about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or any range therein, by weight of the carrier micelle.

For the sol-gel composition and/or the gel-based composition, the second poloxamer, further polymer and/or second poloxamine may be present in an amount from about 0.5% to about 30% or any range therein such as, but not limited to, about 2% to about 15%, or about 2% to about 10% by weight of the sol-gel composition or the gel-based composition. In particular embodiments of the present invention, the second poloxamer, further polymer and/or second poloxamine is present in an amount of about 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or any range therein, by weight of the sol-gel composition or the gel-based composition.

It will be apparent to the skilled artisan that the amount of the second poloxamer and/or poloxamine present in the sol-gel composition may be determined, at least in part, by the desired transition temperature of the sol-gel composition. In some embodiments, decreasing the second poloxamer and/or poloxamine content will increase the transition temperature. Alternatively, increasing the second poloxamer and/or poloxamine content can decrease the transition temperature of the sol-gel composition.

Additionally, it will be appreciated that the gel-based composition typically contains a relatively higher concentration of the of the second poloxamer, further polymer and/or second poloxamine, such that the gel-based composition is suitably of a gel or gel-like consistency at room temperature.

The term “solvent” refers to any liquid capable of maintaining another substance in solution. Examples of solvents include, but are not limited to, organic solvents. It will be apparent to the skilled artisan that the solvent solution may include any appropriate water miscible solvent as are known in the art.

Water miscible solvents are typically miscible with water at a solvent composition less than 50 wt % of the solvent/water mixture. Examples of water miscible solvents include alcohols such as, methanol (approximate boiling point at atmospheric pressure, or b.p.: 65° C.), ethanol (b.p.: 78° C.); ketones such as acetone (b.p.: 56° C.) and various other solvents such as acetonitrile (b.p.: 81° C.), tetrahydrofuran (THF) (b.p.: 66° C.), diethoxymethane (DEM) (b.p.: 87-88° C.), 1,4-dioxane (b.p. 101° C.), and the like. In one particular embodiment, the water miscible solvent is or comprises a ketone, such as acetone, and/or a primary or secondary alcohol, such as methanol, ethanol, propanol (b.p.: 97° C.) and isopropanol (b.p.: 82° C.).

Suitably, the water miscible solvent has a boiling point of less than about 105° C. under standard environmental conditions, such as atmospheric pressure (e.g., about 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40° C. etc and any range therein). In one particular embodiment, the water miscible solvent has a boiling point substantially the same or less than that of water (i.e., about 100° C.) under standard conditions including atmospheric pressure. To this end, the step of substantially removing the water miscible solvent and water from the mixture in (a) to produce a micelle composition, suitably requires that the water miscible solvent evaporates at a rate that is similar to, and preferably quicker than, that of water. In this regard, the water miscible solvent is preferably a liquid at or about room temperature.

In the context of the present disclosure, it will be appreciated that the term “atmospheric pressure” is not to be limited to an exact value for atmospheric pressure, such as 1 atmosphere (760 Torr, or 101.325 kPa) at sea level. Instead, the term “atmospheric pressure” also generally encompasses any pressure that is substantially at, or near atmospheric pressure. Accordingly, the term “atmospheric pressure” can generally encompass a range of pressures from about 720 Torr to about 800 Torr. In one embodiment, the term “atmospheric pressure” refers to 1 atmosphere (760 Torr or 101.325 kPa). For the avoidance of doubt, the term water miscible solvent having a boiling point of less than 105° C. (or the like) at atmospheric pressure refers to a property of the water miscible solvent; the term does not mean that any step is necessarily performed at that pressure.

The term “gel” refers to the state of matter between liquid and solid. As such, a “gel” has some of the properties of a liquid (i.e., the shape is resilient and deformable) and some of the properties of a solid (i.e., the shape is discrete enough to maintain three dimensions on a two dimensional surface).

With regard to the present invention, the sol-gel composition is suitably capable of a sol-gel phase transition at a transition or gelation temperature of about 20° C. to about 40° C. (e.g., about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C.) and any range therein. Preferably, the transition temperature is about 25° C. to about 35° C. More preferably, the transition temperature is about 28° C. to about 34° C.

Accordingly, the sol-gel composition or gel-based composition of the present invention is preferably a solution that is substantially free of particulates or suspended matter particulates at temperatures from about 2° C. to about 30° C. and more particularly about 10° C. to about 25° C. The turbidity or clarity of the compositions of the invention may be determined by any means known in the art, such as visually and turbidimetry. Suitably, the sol-gel composition or the gel-based composition of the present invention has or demonstrates a level of turbidity or clarity of 50 Nephelometric Turbidity Units (NTU) (e.g., 50, 45, 40, 35, 30, 25, 20, 15, 10, 5 NTU or any range therein) or less, more particularly less than 20 NTU and even more particularly less than 10 NTU at temperatures from about 2° C. to about 30° C. and more particularly about 10° C. to about 25° C.

Suitably, the sol-gel composition is a single phase solution, at typical storage temperatures (e.g., about 2° C. to about 20° C.), but when applied to, for example, a mucosal surface of a warm blooded subject (e.g., about 25° C. to about 37° C.) the sol-gel composition is converted to a gel that preferably possesses appropriate rheological and mechanical properties to promote retention at the site of application and ensure reproducible delivery of the therapeutic agent thereto.

The gelation or transition temperature of the sol-gel composition described herein may be determined by any means known in the art, such as with a rheometer or by visual inspection. To this end, it will be appreciated that visual gelation temperatures are typically higher (e.g., about 4-5° C. higher) than equivalent or corresponding rheologically determined gelation temperatures. Accordingly, in particular embodiments the gelation temperatures recited herein are visual gelation temperatures or rheological gelation temperatures. Preferably, the gelation temperatures recited herein are visual gelation temperatures.

In particular embodiments, the thermo-responsive sol-gel composition described herein has a viscosity of less than about 0.15 Pa·s (e.g., 0.15, 0.14, 0.13, 0.12, 0.11, 0.10, 0.09, 0.08, 0.07, 0.06, 0.05 Pa·s etc and any range therein) at about 22° C. and greater than about 0.3 Pa·s (e.g., 0.3, 0.35, 0.4, 0.45, 0.5, 0.55, 0.6, 0.65, 0.7, 0.75, 0.8, 0.85, 0.9, 0.95, 1.0 Pa·s etc and any range therein) at about 30° C., 31° C., 32° C., 33° C., 34° C., 35° C., 36° C., 37° C., or any range therein. It will be appreciated that viscosity may be assessed by any means known in the art, such as with a viscometer or rheometer.

In one embodiment, the thermo-responsive sol-gel composition has a gel strength of greater than about 500 Pa at about 30° C. or at about 35° C. and more particularly greater than about 1000 Pa at about 30° C. or at about 35° C. As used herein, the term “gel strength” refers to the rheology of the gel. These viscoelastic properties of the thermo-responsive sol-gel composition can be determined using standard rheological characterization techniques that will be well known to one having ordinary skill in the art.

As used herein, the terms “approximately” and “about” refer to tolerances or variances associated with numerical values recited herein. The extent of such tolerances and variances are well understood by persons skilled in the art. Typically, such tolerances and variances do not compromise the structure, function and/or implementation of the composition and methods described herein.

In particular embodiments, the carrier micelle described herein is or comprises a nanomicelle. Nanomicelles, including the carrier micelle, have an average size, which refers to the average diameter of the micelle, that may be, for example, no greater than 1000 nanometers, no greater than 500 nanometers, no greater than 200 nanometers, no greater than 100 nanometers, no greater than 75 nanometers, no greater than 50 nanometers, no greater than 40 nanometers, no greater than 25 nanometers, or no greater than 20 nanometers. In certain embodiments, the carrier micelle of the present invention has an average size of between about 10 nm and about 500 nm, or any range therein such as, but not limited to, about 15 nm to about 400 nm, or about 30 nm to about 250 nm. In particular embodiments of the present invention, the carrier micelle has an average size of about 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm or any range therein. In certain embodiments of the present invention, the carrier micelle has an average size of between about 10 nm and about 25 nm.

In other embodiments, the carrier micelle described herein is or comprises a micromicelle. Micromicelles, including the carrier micelle, have an average size, which refers to the average diameter of the micelle, that may be, for example, greater than 1000 nanometers (e.g., 1050 nm, 1100 nm, 1150 nm, 1200 nm, 1250 nm, 1300 nm, 1350 nm, 1400 nm, 1450 nm, 1500 nm, 1600 nm, 1700 nm, 1800 nm, 1900 nm, 2000 nm and any range therein).

By “hydrophobic” is meant the property of a substance, such as a therapeutic agent, that is substantially repellant to water. Accordingly, hydrophobic substances are typically incapable of completely dissolving in an excess of water. Further, a hydrophobic substance tends to be non-polar and, thus, prefers other neutral molecules and non-polar solvents.

As generally used herein, the term “therapeutic agent” refers to a biological or chemical agent that provides a desired biological or pharmacological effect, such as the prevention or treatment of a disease, disorder or condition, when administered to a subject, such as a human or animal. For the sake of example only, the therapeutic agent may be a small molecule, a protein, an antibody or fragment thereof (including a diabody, triabody, or tetrabody), a mimetibody, a mAb, a peptide, an enzyme, a nucleotide, a DNA fragment, an RNA fragment, a plasmid fragment, a nucleotide fragment, or mixtures thereof.

The hydrophobic therapeutic agent provided herein may be any as are known in the art. Suitably, the hydrophobic therapeutic agent is a BCS Class II or Class IV drug.

In particular embodiments, the hydrophobic therapeutic agent is selected from the group consisting of a steroid, an anticancer agent, an antifungal agent, an anti-inflammatory agent, a sex hormone, an immunosuppressant, an antiviral agent, an antibacterial agent, an anti-fibrotic agent, an antihistamine agent, a vitamin, a natural extract, a plant extract and combinations thereof. In one preferred embodiment, the hydrophobic therapeutic agent is or comprises a steroid, such as mometasone furoate, beclomethasone dipropionate, betamethasone, budesonide, ciclesonide, fluticasone furoate, fluticasone propionate and triamcinolone acetonide. In one preferred embodiment, the hydrophobic therapeutic agent is or comprises an antihistamine agent, such as levocabastin. In one preferred embodiment, the hydrophobic therapeutic agent is or comprises an antifungal agent, such as nystatin. In another embodiment, the hydrophobic agent is or comprises a vitamin, such as Vitamin E and/or Vitamin D. In yet another embodiment, the hydrophobic agent is or comprises an immunosuppressant, such as cyclosporin. In a further embodiment, the hydrophobic agent is or comprises an anticancer agent, such as a chemotherapeutic agent (e.g., docetaxel). In another embodiment, the hydrophobic agent is or comprises an anti-inflammatory agent, such as a non-steroidal anti-inflammatory agent (e.g., ketoprofen). In yet another embodiment, the hydrophobic agent is or comprises a natural extract or plant extract, such as curcumin or an extract from the Indian gooseberry (Emblica officinalis). In this regard, it will be appreciated that cosmetic applications of the sol-gel composition and the gel composition described herein are to be encompassed by the present invention.

Based on the above, it is envisaged that the sol-gel, gel-based and micelle compositions described herein may contain a plurality of therapeutic agents, including hydrophobic and hydrophilic therapeutic agents (e.g., 2, 3, 4, 5 etc therapeutic agents). By way of example, the compositions of the invention may contain a hydrophobic therapeutic agent in the carrier micelle and a hydrophilic agent in the second aqueous solution. Additionally, two different formulations of micelle compositions could be made, each containing a different hydrophobic agent and added in the required ratio to the second aqueous solution, which may or may not contain a hydrophilic agent. Furthermore, a micelle composition could contain two or more hydrophobic agents disposed or dispersed within the carrier micelles therein.

For the present invention, the hydrophobic therapeutic agent may be present in the sol-gel composition or the gel-based composition in an amount from about 0.01% to about 20% or any range therein such as, but not limited to, about 0.1% to about 15%, or about 1% to about 10%, or about 2% to about 5% by weight of the sol-gel composition or the gel-based composition. In particular embodiments of the present invention, the hydrophobic therapeutic agent is present in an amount of about 0.01%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, or any range therein, by weight of the sol-gel composition or the gel-based composition. It will be apparent to the skilled artisan that the amount of the therapeutic agent present in the sol-gel composition or the gel-based composition may be determined, at least in part, by the desired dosage, route of administration etc of the therapeutic agent.

Further to the above, the hydrophobic therapeutic agent dispersed or disposed within the hydrophobic core of the carrier micelle may be present in an amount from about 0.02% to about 40% or any range therein such as, but not limited to, about 0.5% to about 25%, or about 10% to about 20% by weight of the carrier micelle. In particular embodiments of the present invention, the hydrophobic therapeutic agent is present in an amount of about 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40% or any range therein, by weight of the carrier micelle. It will be apparent to the skilled artisan that the amount of the therapeutic agent present in the carrier micelle may be determined, at least in part, by the desired concentration of the therapeutic agent in the sol-gel composition or the gel-based composition.

For the present invention, “physiologically acceptable” refers to that which is generally safe, nontoxic and neither biologically nor otherwise undesirable and which is acceptable for pharmaceutical, cosmetic or dietary (human or animal) use. Suitably, a physiologically acceptable solvent, such as for inclusion in the second aqueous solution, can be selected from the group consisting of water, a buffer solution, an acid solution, a basic solution, a salt solution, a saline solution, water for injection, a sugar solution, a glucose solution and any combination thereof. Preferably, the physiologically acceptable solvent is pharmaceutically acceptable for the intended route of administration of the sol-gel composition or the gel-based composition.

As used herein, the term “surfactant” or “surface-active agent” refers to an agent, usually an organic chemical compound that is at least partially amphiphilic (i.e., typically containing a hydrophobic tail group and hydrophilic polar head group). Given their structure, surfactants are generally capable of lowering the surface tension (or interfacial tension) between two liquids or between a liquid and a solid.

The surfactant described herein may be any as are known in the art and is suitably selected from the group consisting of a polyoxyethylated sorbitan fatty ester (i.e., a polysorbate—e.g., Tween 20 (polyoxyethylene sorbitan monolaurate), Tween 40 (polyoxyethylene sorbitan monopalmitate), Tween 60 (polyoxyethylene sorbitan monostearate), Tween 80 (polyoxyethylene sorbitan monooleate), a polyoxyethylated glycol monoether (e.g., macrogol 15 hydroxystearate, polyethylene glycol (15)-hydroxystearate, polyoxyethylated 12-hydroxystearic acid (Kolliphor HS15, Solutol)), a polyoxyethylated glyceride, n-dodecyl tetra (ethylene oxide), a polyoxyethylated fatty acid, a polyoxyethylated castor oil (e.g. Cremophor EL (CrEL) or Kolliphor EL), a sucrose ester, a lauroyl macroglyceride, a poloxamer, a polyglycolyzed glyceride and combinations thereof. In one particular embodiment, the surfactant is or comprises a polyoxyethylene sorbitan C15-21 alkene, such as polyoxyethylene (20) sorbitan monooleate (e.g., Tween 80). Preferably, the surfactant is a liquid at room temperature (e.g., at about 20° C. to about 25° C.). Without intending to be limited by theory, it is believed that the surfactant with its amphiphilic structure is responsible for further stabilising the therapeutic agent-filled carrier micelles by associating with complementary components of the poloxamer and/or the poloxamine, and enhancing the retention of the therapeutic in the micelles when dispersed or disposed in the sol-gel composition or the gel-based composition, so as to improve stability and efficacy thereof.

It is envisaged that one or both of the solvent solution and the first aqueous solution can include the surfactant dissolved or dispersed therein. In one particular embodiment, the first aqueous solution comprises the surfactant. In an alternative embodiment, the solvent solution comprises the surfactant.

For the first aqueous solution and/or the solvent solution, the surfactant may be present in an amount from about 0.25% to about 20% or any range therein such as, but not limited to, about 2% to about 15%, about 2% to about 10%, or about 5% to about 10% by weight of the first aqueous solution and/or the solvent solution. In particular embodiments of the present invention, the surfactant is present in an amount of about 0.25%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90% or any range therein, by weight of the first aqueous solution and/or the solvent solution.

In some embodiments, the surfactant is present in an amount of about 0.05% to about 70% (e.g., about 0.05%, 0.1%, 0.2%, 0.3%, 0.4%, 0.5%, 1.0%, 1.5%, 2.0%, 2.5%, 3.0%, 3.5%, 4.0%, 4.5%, 5.0%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%), or any range therein, by weight of the carrier micelle, and in some embodiments from about 5% to about 50% or from about 20% to about 50% surfactant by weight of the carrier micelle. In some embodiments, increasing the surfactant content can delay or increase the transition or gelation time of the sol-gel composition, whilst decreasing the surfactant content can decrease the transition or gelation time of the sol-gel composition. In other embodiments, decreasing the surfactant content will decrease the transition temperature. Alternatively, increasing the surfactant content can increase the transition temperature of the sol-gel composition.

Suitably, the surfactant has a Hydrophile—Lipophile Balance (HLB) number between about 5 and about 20 (e.g., about 5, 6, 7, 8, 9, 10, 11, 12, 13, 14, 15, 16, 17, 18, 19, 20 and any range therein). As will be understood by the skilled artisan, HLB is a numerical system used to describe the relationship between the water-soluble and oil-soluble parts of a nonionic surfactant. HLB numbers typically range from 1 to 30. For example, if a surfactant has an HLB=1, it is considered very oil soluble, while a surfactant with an HLB=15 is considered to be water soluble. The HLB number is also considered to be a measure of the % ethoxylation (EO) of the respective surfactant. Hydrophilic surfactants are water-soluble and are used for solubilization, detergency, and for products that will be readily miscible with water.

With respect to step (a) of the present method, the mixture of the first aqueous solution and the solvent solution may be treated by any means or method known in the art so as to incorporate the hydrophobic therapeutic agent into the hydrophobic polymer core of the carrier micelle. By way of example, the mixture of the first aqueous solution and the solvent solution may be subjected to stirring, heating, ultrasonic treatment, vortexing, solvent evaporation, dialysis or any combination thereof to achieve this end.

It is envisaged that the step of removing the water miscible solvent and water from the mixture in (a) may be performed by any means in the art, inclusive of the same or different means. By way of example, the water miscible solvent and/or water may be at least partly removed by rotary evaporation, flushing with air or an inert gas at high pressure, lyophilization or freeze drying, or distillation under reduced or negative pressure. As such, removing the water miscible solvent and removing water from the mixture in step (a) of the present method may include separate steps respectively. As used here, the terms “high pressure” refers to a pressure higher than atmospheric pressure (or 1 atmosphere, 101.325 kPa or 760 Torr). Similarly, the term “reduced pressure” refers to a pressure lower than atmospheric pressure (or 1 atmosphere, 101.325 kPa or 760 Torr).

Suitably, the step of removing the water miscible solvent is performed at a temperature from about 25° C. to about 35° C. (e.g., about 25, 26, 27, 28, 29, 30, 31, 32, 33, 34, 35° C. and any range therein). In one particular embodiment, the step of removing the water miscible solvent is performed at a temperature from about 32° C. to about 34° C. Such cooler temperatures than typically used in other methods of forming micelles (e.g., thin film method), advantageously allow for the better incorporation of the hydrophobic therapeutic agent within the carrier micelles and hence better stability. Additionally, certain hydrophobic therapeutic agents, such as vitamins, are temperature sensitive and these lower temperatures for solvent removal prevent or minimise any degradation thereof.

In certain embodiments, the sol-gel composition or the gel-based composition further comprises an appropriate pharmaceutically-acceptable carrier, diluent or excipient. Preferably, the pharmaceutically-acceptable carrier, diluent or excipient is suitable for administration to mammals, and more preferably, to humans.

By “pharmaceutically-acceptable carrier, diluent or excipient” is meant a solid or liquid filler, diluent, carrier or encapsulating substance that may be safely used in systemic administration. Depending upon the particular route of administration, a variety of excipients or carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates, and pyrogen-free water.

In regard to embodiments that are to be topically applied to, for example, a mucous membrane or a portion of skin, the composition may include one or more humectants, emollients or soothing agents. Non-limiting examples of humectants include propylene glycol, hexylene glycol, butylene glycol, aloe vera gel, alpha hydroxy acids such as lactic acid, egg yolk, egg white, glyceryl triacetate, honey, lithium chloride, molasses, polymeric polyols such as polydextrose, quillaia, sodium hexametaphosphate E452i, sugar alcohols (sugar polyols) such as glycerol, glycerine, sorbitol, xylitol and maltitol, and urea.

In addition to the above, the sol-gel composition and the gel-based composition described herein may further include one or more of a preservative (e.g., propyl paraben), a mechanical strength enhancer (e.g., hydroxypropyl methyl cellulose; HPMC E4M), a mucoadhesive (e.g., chitosan) and a thickening agent/emulsifier (e.g., HPMC).

Each of the aforementioned excipients (e.g., humectants, emollients, soothing agents, preservatives, mechanical strength enhancers, mucoadhesives, thickening agents, emulsifiers etc) may be included in the sol-gel composition or the gel-based composition at in an amount of about 0.05% to about 10% (e.g., about 0.05%, 0.1%, 0.2%, 0.3%, 0.4%, 0.5%, 1.0%, 1.5%, 2.0%, 2.5%, 3.0%, 3.5%, 4.0%, 4.5%, 5.0%, 6%, 7%, 8%, 9%, 10%), or any range therein, by weight of the sol-gel composition or the gel-based composition. Generally, such excipients can be included in the second aqueous solution and/or added separately to the sol-gel composition or the gel-based composition described herein.

In certain embodiments, the composition may include one or more pH-adjusting agents. For example, hydrochloric acid solutions or sodium hydroxide solutions may be added to adjust the pH of the composition. In certain embodiments, the pH of the composition is from about 5.0 to about 8.2 (e.g., about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2 and any range therein).

A useful reference describing pharmaceutically acceptable carriers, diluents and excipients is Remington's Pharmaceutical Sciences (Mack Publishing Co. NJ USA, 1991).

In a further aspect, the invention provides a thermo-responsive sol-gel composition prepared by the method of first mentioned aspect.

In another aspect, the invention relates to a gel-based composition prepared by the method of the second mentioned aspect.

In a related aspect, the invention resides in a micelle composition prepared by the method of the third mentioned aspect.

In another aspect, the invention provides a thermo-responsive sol-gel composition or a gel-based composition for therapeutic use comprising:
an aqueous solution;
a carrier micelle disposed within the aqueous solution and including a poloxamer and/or a poloxamine and a surfactant and having a hydrophobic core; and
a hydrophobic therapeutic agent disposed within the hydrophobic core of said carrier micelle.

Suitably, the thermo-responsive sol-gel composition or the gel-based composition is prepared according to the method described herein. In this regard, the aqueous solution suitably comprises a further poloxamer and/or poloxamine, such as those hereinbefore described, that may include a sol-gel transition property as required.

Suitably, the thermo-responsive sol-gel composition has a gelation temperature below about 35° C. More particularly, the gelation temperature can be between about 20° C. to about 32° C.

In one embodiment, the thermo-responsive sol-gel composition has a viscosity of less than about 0.15 Pa·s at about 22° C. and greater than about 0.3 Pa·s at about 30° C. or about 35° C.

In other embodiments, the thermo-responsive sol-gel composition has a gel strength of greater than about 500 Pa at about 30° C. or at about 35° C. and more particularly greater than about 1000 Pa at about 30° C. or at about 35° C.

Suitably, the surfactant can be that previously described herein such as a polyoxyethylated sorbitan fatty ester, a polyoxyethylated glycol monoether, a polyoxyethylated glyceride, n-dodecyl tetra (ethylene oxide), a polyoxyethylated fatty acid, a polyoxyethylated castor oil, a sucrose ester, a lauroyl macroglyceride, a polyglycolyzed glyceride and any combination thereof. In one particular embodiment, the surfactant is or comprises polyoxyethylene sorbitan monooleate.

Suitably, the hydrophobic therapeutic agent is that previously provided herein, such as a steroid, an anticancer agent, an antifungal agent, an anti-inflammatory agent, a sex hormone, an immunosuppressant, an antiviral agent, an antibacterial agent, an anti-fibrotic agent, an antihistamine agent, a vitamin, a plant extract and any combination thereof.

Similarly, the poloxamer and/or poloxamine may be that as previously provided herein, such as P407.

In a further aspect, the invention resides in a method of administering a hydrophobic therapeutic agent to a subject, the method including the step of administering the thermo-responsive sol-gel composition, the gel-based composition and/or the micelle composition described herein to the subject.

The term “subject” includes warm blooded subjects, and in particular human and veterinary subjects. For example, administration to a subject can include administration to a human subject or a veterinary subject. Preferably, the subject is a human. However, therapeutic uses according to the invention may also be applicable to mammals such as domestic and companion animals, performance animals such as horses, livestock, and laboratory animals.

By “administration” is intended the introduction of a composition (e.g., a thermo-responsive sol-gel composition, the gel-based composition and/or a micelle composition), into a subject by a chosen route.

Any safe route of administration may be employed for providing a subject with the sol-gel composition, the gel-based composition and/or micelle composition described herein. For example, oral, rectal, parenteral, sublingual, buccal, intravenous, intra-articular, intra-muscular, intra-dermal, subcutaneous, inhalational, intra-nasal, intraocular, intraperitoneal, intracerebroventricular, transdermal, and the like may be employed. In one particularly preferred embodiment, the sol-gel composition is adapted for administration to a mucosal membrane, including, but not limited to intra-nasal administration, intrarectal administration and intravaginal administration, such as via a catheterized syringe or nasal spray.

The sol-gel composition, the gel-based composition and/or micelle composition provided herein may be administered in a manner compatible with the dosage formulation and the hydrophobic therapeutic agent therein, and in such amount as is pharmaceutically/therapeutically-effective. The dose administered to a subject, in the context of the present invention, should be sufficient to effect a beneficial response (e.g. a reduction in a symptom of a disease, disorder or condition) in a subject over an appropriate period of time. The quantity of the sol-gel composition, the gel-based composition and/or micelle composition to be administered may depend on the subject to be treated, inclusive of the age, sex, weight and general health condition thereof, factors that will depend on the judgement of a practitioner of ordinary skill in the art.

In yet a further aspect, the invention provides a method of preventing and/or treating a disease, disorder or condition in a subject, including the step of administering to the subject a therapeutically effective amount of the thermo-responsive sol-gel composition, the gel-based composition and/or the micelle composition described herein to thereby prevent and/or treat the disease, disorder or condition.

In yet another further aspect, the invention resides in a thermo-responsive sol-gel composition, a gel-based composition or a micelle composition described herein for use in preventing and/or treating a disease, disorder or condition in a subject.

In a related aspect, the inventions provides the use of the thermo-responsive sol-gel composition, the gel-based composition and/or the micelle composition described herein, in the manufacture of a medicament for preventing and/or treating a disease, disorder or condition in a subject.

As used herein, “treating” (or “treat” or “treatment”) refers to a therapeutic intervention that ameliorates a sign or symptom of the disease, disorder or condition after it has begun to develop. The term “ameliorating”, with reference to a disease, disorder or condition, refers to any observable beneficial effect thereto of the treatment. The beneficial effect can be determined using any methods or standards known to the ordinarily skilled artisan.

As used herein, “preventing” (or “prevent” or “prevention”) refers to a course of action (such as administering a therapeutically effective amount of the thermoresponsive sol-gel composition, the gel-based composition and/or micelle composition described herein) initiated prior to the onset of a symptom, aspect, or characteristic of the disease, disorder or condition so as to prevent or reduce the symptom, aspect, or characteristic. It is to be understood that such preventing need not be absolute to be beneficial to a subject. A “prophylactic” treatment is a treatment administered to a subject who does not exhibit signs of the disease, disorder or condition or exhibits only early signs for the purpose of decreasing the risk of developing a symptom, aspect, or characteristic of the disease, disorder or condition.

The term “therapeutically effective amount” describes a quantity of a specified agent sufficient to achieve a desired effect in a subject being treated with that agent. For example, this can be the amount of the sol-gel composition, the gel-based composition and/or micelle composition necessary to reduce, alleviate and/or prevent the disease, disorder or condition. In some embodiments, a “therapeutically effective amount” is sufficient to reduce or eliminate a symptom of the disease, disorder or condition. In other embodiments, a “therapeutically effective amount” is an amount sufficient to achieve a desired biological effect, for example an amount that is effective to decrease inflammation and/or pain associated with the disease, disorder or condition.

Ideally, a therapeutically effective amount of an agent is an amount sufficient to induce the desired result without causing a substantial cytotoxic effect in the subject. The effective amount of an agent, for example a thermo-responsive sol-gel composition, a gel-based composition and/or a micelle composition, useful for reducing, alleviating and/or preventing a disease, disorder or condition, such as a respiratory disease, disorder or condition will be dependent on the subject being treated, the type and severity of any associated disease, disorder and/or condition, and the manner of administration of the therapeutic composition.

A therapeutically effective amount of the sol-gel composition, the gel-based composition and/or the micelle composition described herein may be administered in a single dose, or in several doses, for example daily, during a course of treatment. However, the frequency of administration is dependent on the preparation applied, the subject being treated, the severity of the disease, disorder or condition, and the manner of administration of the therapy or composition.

Referring to the above aforementioned aspects, the disease, disorder or condition suitably is or comprises a respiratory disease, disorder or condition. In particular embodiments, the respiratory disease, disorder or condition is selected from the group consisting of rhinitis, infectious rhinitis, allergic rhinitis, sinusitis, asthma, chronic obstructive pulmonary disease (COPD), bronchitis and any combination thereof. In other embodiments, the disease, disorder or condition suitably is or comprises a cancer, such as a throat cancer, a rectal cancer, a cervical cancer, a vaginal cancer, an endometrial cancer, an ovarian cancer or a skin cancer (e.g., melanoma). In other embodiments, the disease, disorder or condition is or comprises a skin, mucosal and/or topical disease, disorder or condition (e.g., atopic dermatitis, acne, eczema, psoriasis, vitiligo, candidiasis, haemorrhoids). In certain embodiments, the disease, disorder or condition is or comprises an ear or otic disease, disorder or condition (e.g., otitis externa, a middle or inner ear infection and inflammation). In a further embodiment, the disease, disorder or condition is or comprises an ophthalmic disease, disorder or condition (e.g., glaucoma, conjunctivitis).

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

Any reference to publications cited in this specification is not an admission that the disclosures constitute common general knowledge in Australia.

In order that the invention may be more readily understood and put into practice, one or more preferred embodiments thereof will now be described, by way of example only.

### EXAMPLES

### Example 1

Example 1 provides a method of preparing a thermoresponsive sol-gel composition comprising the hydrophobic therapeutic agent mometasone furoate (MF) solubilized in a micelle component, wherein the micelle component further includes a surfactant.

### Materials:

Mometasone furoate (MF), Poloxamer (P407), polysorbate 80 (Tween® 80), hydroxypropyl methyl cellulose (HPMC E4M), low molecular weight chitosan (50,000-190,000 Da, 75-85% deacetylated), glycerin, propyl paraben, acetone, acetic acid, sodium hydroxide (NaOH), deionized water.

### Method:

Step 1: Preparation of MF-loaded polymeric micelles:
MF loaded polymeric micelles were first prepared by adapting the solvent evaporation method:
10 mg of MF was added to 10 mL of acetone forming a clear drug solution (equivalent to 0.1% w/w MF of the final sol-gel formulation)
the aforementioned drug solution was mixed with 1.1 g of P407 (equivalent to 11% w/w of the final sol-gel formulation) and 0.2-1 g of Tween® 80 (equivalent to 2-10% w/w of the final sol-gel formulation) in 8 mL deionized water
the mixture was stirred thoroughly (400 rpm) for 30 minutes followed by sonication for 2 minutes at room temperature
acetone was completely removed by rotary evaporation (34° C. water bath, for 1 h) to obtain a dual-polymeric MF-micellar solution
the resultant solution was then filtered (0.22 μm) to remove traces of unentrapped/undissolved MF
the eluted MF-micellar solution was lyophilized, and stored at 2-8° C. until required
   Step 2: Preparation of a single phase, thermo-responsive sol-gel system containing MF-micelles:
0.35 g of P407 (equivalent to 3.5% w/w of the final sol-gel formulation) was added to a minimum volume (2-3 mL) of cold deionized water and mixed thoroughly (400 rpm) for 45 min at 2-8° C., and then hydrated overnight at 2-8° C.
separately, a stock solution of 1% w/w chitosan in acetic acid (1% v/v) was prepared
20 mg of HPMC (equivalent to 0.2% w/w of the final sol-gel formulation), 10 mg of chitosan solution (equivalent to 0.1% w/w of the final sol-gel formulation), 5 mg of propyl paraben (equivalent to 0.05% w/w of the final sol-gel formulation) and 0.3 g of glycerin as humectant/soothing agent (equivalent to 3% w/w of the final sol-gel formulation), were added to the cold poloxamer solution
the lyophilized MF-micelle powder was added slowly to this polymeric solution with continuous stirring (300 rpm) at room temperature
Finally, the formulation was adjusted to pH 6 with 0.1 M HCl or 0.1 N NaOH, and made up to 10 g (˜10 mL) with deionized water and stored overnight at room temperature until required.

### Results and Conclusions

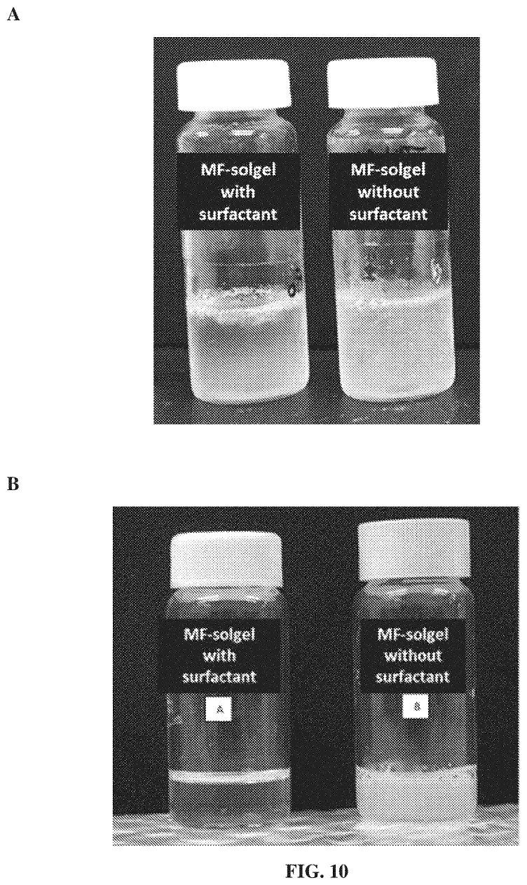

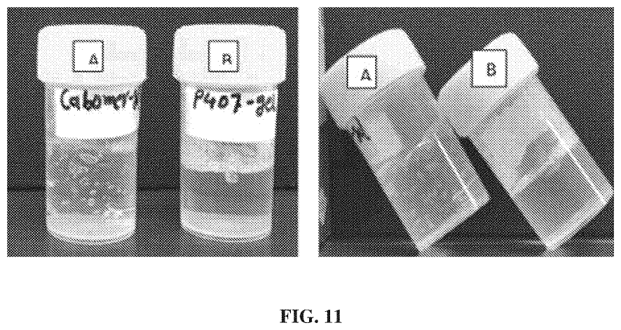

It is postulated that the addition of surfactant acts at the core and shell-level of the therapeutic agent-filled micelles, acting as a ‘sealant’ to trap the therapeutic agent within the P407-containing micelles, which should in turn prevent drug leaching out into the sol-gel base. The visual appearance of the sol-gel composition prepared with and without polysorbate 80 is strikingly different (see FIG. 10), and we believe a lack of polysorbate 80 causes free migration of P407 from drug-filled micelles into the sol-gel composition, leading to a slightly ‘cloudy’ or ‘hazy’ formulation over time, and we expect that this would eventually lead to drug also leaching out. Through addition of polysorbate 80, the drug-filled micelles are effectively ‘sealed’ and the sol-gel remains clear upon storage. Interestingly, polysorbate 80 is also a permeation enhancer, so it is believed this could also have a positive effect from a drug delivery perspective, by promoting therapeutic agent-micelle permeation into surrounding mucosal tissue.

MF is an anti-inflammatory steroid used clinically for the treatment of nasal symptoms of seasonal and perennial allergic rhinitis; prophylaxis of nasal symptoms of seasonal allergic rhinitis; relief of nasal congestion associated with seasonal allergic rhinitis; treatment of nasal polyps. MF is classified as being “practically insoluble” in aqueous media e.g. in gel-based systems. The developed MF-containing nanomicellar formulation is a homogenous system at storage temperature (2-8° C.), where solute (MF containing micelles, polymers & other excipients) and solvent constitute only one phase (single-phase) and are uniformly distributed throughout a liquid with no visible/apparent boundary between the dispersed solutes and aqueous polymer base. At nasal temperature (circa. 34° C.), formulations are converted to a gel possessing appropriate rheological and mechanical properties that avoid anterior or posterior leakage, promote retention, and ensure reproducible drug delivery to local mucosal tissue.

Uniformity of nasal formulations is an important criteria for accurate drug dosage delivery. The liquid-like viscosity of our formulation is expected to promote ease of drug administration to the sinuses, via a catheterized syringe (during surgery) or nasal spray (for patient self-administration)-based system.

The sol-gel formulation of the present example transforms and maintains the drug in a soluble state, which is expected to enhance drug absorption at the targeted nasal mucosal site, and allowing for reduced dosages in nasal formulations (c.f. existing nasal suspensions)

The liquid-like properties at storage temperature provide ease of administration, while there is rapid (less than 60 secs) and full conversion to gel-form where MF-nanomicelles are homogenously distributed throughout the formulation.

The optimized ‘recipe’ of the MF-containing sol-gels provide for ideal rheological, mechanical and mucoadhesive properties when in the gel form, with sustained drug release properties that would more efficiently and effectively treat symptoms of mucosal inflammation, such as sinusitis, and support rapid wound healing in case of post-operative inflammation of any mucosal tissue (e.g. rectal, vaginal, oromucosal, throat, oesophageal, sinus cavity).

Glycerin is present as a soothing agent and humectant providing emollient-based relief to inflamed nasal mucosal tissues in CRS and pre/post-operative sinusitis.

Importantly, the developed MF-containing sol-gel formulations address as series of shortfalls with existing clinically available nasal solutions and suspensions, including anterior and posterior leakage, increased dosing frequency and drug dose, unpredictable drug absorption, which adversely affect patient compliance with currently marketed conventional MF formulations.

### Example 2

In the present example, mometasone furoate (MF)-sol-gels were prepared via four different processes or methodologies as outlined below.

### Method 1.

This comprised two distinct phases, wherein in Phase 1 the MF-micelles were first prepared using the total concentrations of poloxamer and surfactant, which after lyophilisation was combined with the remaining excipients in Phase 2 (HPMC, chitosan, glycerin & propyl paraben) and adjusted to pH 5 then made to volume.

### Phase 1

1.8 g of P407 (equivalent to 18% w/w of the final sol-gel formulation)+0.8 g of Tween® 80 (equivalent to 8% w/w of the final sol-gel formulation) were mixed and stirred in 7 mL water at room temperature. Separately, 10 mg of MF (equivalent to 0.1% w/w MF of the final sol-gel formulation) was dissolved in 10 mL acetone.
This drug solution was added to the polymeric-surfactant solution in a round bottomed flask, then mixed thoroughly (400 rpm) for 15 minutes at room temperature using a magnetic stirrer.
Finally, acetone was removed by rotary evaporation at 32-34° C. to obtain a polymeric micellar solution and the filtrate (micellar solution) was lyophilised and stored at 2-8° C. for further use.

### Phase 2

Optimised concentrations of HPMC (20 mg, equivalent to 0.2% w/w of the final sol-gel formulation), chitosan solution (10 mg, equivalent to 0.1% w/w of the final sol-gel formulation), 10 mg, equivalent to 0.1% w/w of the final sol-gel formulation), glycerin as humectant/soothing agent (0.3 g, equivalent to 3% w/w of the final sol-gel formulation) and propyl paraben (2 mg, equivalent to 0.02% w/w of the final sol-gel formulation), were mixed thoroughly (400 rpm) in water (˜2-3 mL) for 45-60 min and left to fully hydrate overnight (12-14 h) at 2-8° C.
Finally, the lyophilised MF-micelles as dry powder was slowly added to the fully hydrated polymer-excipient mixture, which was then adjusted to pH≈5 with 0.1 M HCl or 0.1 N NaOH, and made up to 10 g (≈10 mL) with Milli-Q water.
The final MF micelle-infused sol-gel formulations were stored at cold temperature (2-8° C.) until required for further use.

### Method 2

This method replicated that method described by Dong Wuk Kim et al, Int J Nanomed 2014.
10 mg of MF (equivalent to 0.1% w/w of the final sol-gel formulation)+0.8 g of Tween® 80 (equivalent to 8% w/w of the final sol-gel formulation) were mixed together at 2-8° C.
Separately, optimised concentrations of HPMC (20 mg, equivalent to 0.2% w/w of the final sol-gel formulation), chitosan solution (10 mg, equivalent to 0.1% w/w of the final sol-gel formulation), glycerin as humectant/soothing agent (0.3 g, equivalent to 3% w/w of the final sol-gel formulation) and propyl paraben (2 mg, equivalent to 0.02% w/w of the final sol-gel formulation), were added to a cold solution of 1.8 g of P407 (equivalent to 18% w/w of the final sol-gel formulation) in water, and mixed thoroughly at 2-8° C.
MF-surfactant mixture was added to poloxamer solution and thoroughly mixed. The final formulation was stored at cold temperature (2-8° C.) until required for further use.

### Method 3:

This was prepared using a one pot/phase method, wherein the MF-micelles were prepared using the total concentrations of poloxamer and surfactant and the HPMC, chitosan, glycerin & propyl paraben, which after lyophilisation and adjustment to pH 5 were then made to volume with water.
1.8 g of P407 (equivalent to 18% w/w of the final sol-gel formulation)+0.8 g of Tween® 80 (equivalent to 8% w/w of the final sol-gel formulation) were mixed and stirred in 7 mL of water at room temperature.
Next, HPMC (20 mg, equivalent to 0.2% w/w of the final sol-gel formulation), chitosan solution (10 mg, equivalent to 0.1% w/w of the final sol-gel formulation), glycerin as humectant/soothing agent (0.3 g, equivalent to 3% w/w of the final sol-gel formulation) and propyl paraben (2 mg, equivalent to 0.02% w/w of the final sol-gel formulation) were added to the P407-Tween® 80 solution, and mixed thoroughly (400 rpm) for 45-60 min. Separately, 10 mg of MF (equivalent to 0.1% w/w MF of the final sol-gel formulation) was dissolved in 10 mL acetone.
This drug solution was added to the above polymeric solution in a round bottomed flask, the mixed thoroughly (400 rpm) for 15 minutes at room temperature using a magnetic stirrer.
Acetone was removed by rotary evaporation at 32-34° C. to obtain a polymeric micellar solution and lyophilised.
Finally, the lyophilised MF-micelles as dry powder was slowly added to Milli-Q water, which was then adjusted to pH≈5 with 0.1 M HCl or 0.1 N NaOH, and made up to 10 g (≈10 mL) with Milli-Q water.

### Method 4

This method included two distinct phases, wherein in Phase 1 the MF micelles were first prepared using 12% w/w of poloxamer+8% w/w surfactant (these % w/w amounts being based on the final sol-gel formulation), which after lyophilisation was combined with the remaining excipients in Phase 2 (P407, HPMC, chitosan solution, glycerin & propyl paraben) and adjusted to pH 5 then made to volume.

### Phase 1

First, 1.2 g of P407 (equivalent to 12% w/w of the final sol-gel formulation) and 0.8 g of Tween® 80 (equivalent to 8% w/w of the final sol-gel formulation) were added to a round bottomed flask and dissolved in 7 mL of distilled water, then mixed thoroughly (400 rpm) at room temperature for 30 minutes using a magnetic stirrer at room temperature.
Separately, 10 mg of drug (MF, equivalent to 0.1% w/w MF of the final sol-gel formulation) was dissolved in 10 mL of acetone.
Next, this drug solution was added to the polymeric-surfactant solution in a round bottomed flask, the mixed thoroughly (400 rpm) for 15 minutes at room temperature using a magnetic stirrer.
Finally, acetone was removed by rotary evaporation at 32-34° C. to obtain a polymeric micellar solution, and the filtrate (micellar solution) was lyophilised and stored at 2-8° C. for further use.

### Phase 2

First, optimised concentrations of P407 (0.6 g, equivalent to 6% w/w of the final sol-gel formulation) were added to a required amount of cold Milli-Q water (˜2-3 mL) and this solution was mixed thoroughly (400 rpm) for 45 min, at 2-8° C.
Optimised concentrations of HPMC (20 mg, equivalent to 0.2% w/w of the final sol-gel formulation), chitosan solution (10 mg, equivalent to 0.1% w/w of the final sol-gel formulation), glycerin as humectant/soothing agent (0.3 g, equivalent to 3% w/w of the final sol-gel formulation) and propyl paraben (2 mg, equivalent to 0.02% w/w of the final sol-gel formulation), were added to the cold P407 solution which was mixed thoroughly (400 rpm) for 45-60 min and left to fully hydrate overnight (12-14 h) at 2-8° C.
Finally, the lyophilised MF-micelles as dry powder was slowly added to the fully hydrated polymer-excipient mixture, which was then adjusted to pH≈5 with 0.1 M HCl or 0.1 N NaOH, and made up to 10 g (≈10 mL) with Milli-Q water.
The final MF micelle-infused sol-gel formulations were stored at cold temperature (2-8° C.) until required for further use.
   The total concentration of P407 in the sol-gel was 18% w/w (12% w/w from phase 1 and 6% w/w from phase 2)

### Method 4A

This method was adapted from Method 4 above, but with no lyophilisation in Phase 1. Thus, in Phase 1 the MF-micelles were first prepared using 12% w/w of poloxamer and 8% w/w surfactant (these % w/w amounts being based on the final sol-gel formulation), which was (partially) evaporated to remove acetone. The resultant micellar solution was then directly combined with the remaining excipients in Phase 2 (P407, HPMC, chitosan solution, glycerin & propyl paraben) and adjusted to pH 5 then made to volume.

### Phase 1

First, 1.2 g of P407 (equivalent to 12% w/w of the final sol-gel formulation) and 0.8 g of Tween® 80 (equivalent to 8% w/w of the final sol-gel formulation) were added to a round bottomed flask and dissolved 20 in 5 mL of distilled water, then mixed thoroughly (400 rpm) at room temperature for 20 minutes.
Separately, 10 mg of MF (equivalent to 0.1% w/w MF of the final solgel formulation) was dissolved in 5 mL of acetone.
Next, this drug solution was added to the polymeric-surfactant solution in a round bottomed flask, the mixed thoroughly (400 rpm) for 15 minutes at room temperature.
Finally, acetone was removed by rotary evaporation at 32-34° C. to obtain a polymeric micellar solution, and the filtrate (micellar solution) was used directly in phase 2.

### Phase 2

First, 0.6 g of P407 (equivalent to 6% w/w of the final sol-gel formulation) was added to a required amount of cold Milli-Q water (˜2-3 mL) and this solution was mixed thoroughly (400 rpm) for 45 min, at 2-8° C.
Optimised concentrations HPMC (20 mg, equivalent to 0.2% w/w of the final sol-gel formulation), chitosan solution (10 mg, equivalent to 0.1% w/w of the final sol-gel formulation), glycerin as humectant/soothing agent (0.3 g, equivalent to 3% w/w of the final sol-gel formulation) and propyl paraben (2 mg, equivalent to 0.02% w/w of the final sol-gel formulation) were added to the cold P407 solution which was mixed thoroughly (400 rpm) for 45-60 min and left to fully hydrate overnight (12-14 h) at 2-8° C.
Finally, the drug-micelle solution was added to the fully hydrated polymer-excipient mixture, which was then adjusted to pH≈5 with 0.1 M HCl or 0.1 N NaOH, and made up to 10 g (≈10 mL) with Milli-Q water.
The final drug micelle-infused sol-gel formulations were stored at cold temperature (2-8° C.) until required for further use.

TABLE 2
Size and polydispersity index of MF-loaded micelles (reconstituted
in water) prepared using different methods on day of preparation.

| Formulation | Size (nm) | PDI |
|---|---|---|
| Method 1 | 24.32 | 0.386 |
| Method 2 | 186.11 | 1.328 |
| Method 3 | 38.64 | 0.431 |
| Method 4 | 17.07 | 0.197 |
| Method 4A | 137.56 | 0.768 |

TABLE 3
NTU value, gelation temperature, viscosity and gel strength of
MF-loaded sol-gel formulations prepared using different methods.

| | | | | Gel |
|---|---|---|---|---|
| | | | Viscosity | strength |
| | NTU | Gelation temperature | (Pa · s) | (Pa) |
| Formulation | value | (° C.) | (30° C.) | (30° C.) |
| Method 1 | 49.32 | No sign of gelation | 0.123 | 0.0689 |
| | | by 40° C. | | |
| Method 2 | 220.18 | Transition from | 0.103 | 0.787 |
| | | sol-to-gel | | |
| | | incomplete by 40° C., | | |
| | | and confirmed by | | |
| | | unacceptable target | | |
| | | viscosity and gel | | |
| | | strength\hat{ } | | |
| Method 3 | 78.67 | Transition from | 0.157 | 0.418 |
| | | sol-to-gel | | |
| | | incomplete by 40° C., | | |
| | | and confirmed by | | |
| | | unacceptable target | | |
| | | viscosity and gel | | |
| | | strength\hat{ } | | |
| Method 4 | 24.67 | 21.94° C. | 0.601 | 14010.4 |
| Method 4A^{#} | 121.89 | 28.90° C. | 0.474 | 4974.5 |

*NTU value of blank sol-gel = 14.2. Freshly prepared formulations were stored at 2-8° C. for 6 hrs, followed by 1 hr at room temperature prior to NTU (turbidity) measurements being taken. However, gelation temperature, viscosity and gel strength were measured immediately after 6 hrs storage at 2-8° C.
^{#}No lyophilisation performed at Phase 1, with micellar solution (acetone removed) directly incorporated into sol-gel base.
\hat{ }Target viscosity and gelation values were >0.4 Pa · s and >500 Pa, respectively at/above the target gelation temperature (30° C.), representing complete gelation of the formulation (as noted visually).

### SUMMARY

For Method 1 no crossover point was detected even at 40° C. (blue line). For Methods 2 & 3 although a cross-over point was detected, this was not sustained resulting in an unstable sol-gel state alongside highly turbid formulations in both instances where target values for viscosity and gel strength were also not achieved for either method. For Method 4 the cross over point (gelation) occurred at ≈22° C., in the absence of turbidity, with target values for viscosity & gel strength achieved/exceeded. In the case of Method 4A, where the lyophilisation step in Phase 1 was omitted (c.f. Method 4), a highly turbid formulation resulted, and with a considerable different (elevated) gelation temperature (≈7° C. higher c.f. Method 4).

### Example 3

Three distinct sol-gels infused with vitamin D, vitamin E and cyclosporine sol-gels were prepared using a process developed consistent with Example 2 (Method 4).
The process is summarised below:
Process overview: This comprised two distinct phases, wherein Phase 1 the vitamin or cyclosporine-micelles were first prepared using 12% w/w of poloxamer and 8% w/w surfactant (these % w/w amounts being based on the final sol-gel formulation), which after lyophilisation was combined with the remaining excipients in Phase 2 (P407, HPMC, chitosan solution, glycerin & propyl paraben) and adjusted to pH 5 then made to volume.

### Phase 1

First, 1.2 g of P407 (equivalent to 12% w/w of the final sol-gel formulation) and 0.8 g of Tween® 80 (equivalent to 8% w/w of the final sol-gel formulation) were added to a round bottomed flask and dissolved in 7 mL of distilled water, then mixed thoroughly (400 rpm) at room temperature for 30 minutes using a magnetic stirrer at room temperature.
Separately, 10 mg of drug (vit E, vit D or cyclosporine, equivalent to 0.1% w/w of the final sol-gel formulation) was dissolved in 10 mL of acetone.
Next, this drug solution was added to the polymeric-surfactant solution in a round bottomed flask, then mixed thoroughly (400 rpm) for 15 minutes at room temperature using a magnetic stirrer.
Finally, acetone was removed by rotary evaporation at 32-34° C. to obtain a polymeric micellar solution, and the filtrate (micellar solution) was lyophilised and stored at 2-8° C. for further use.

### Phase 2

First, optimised concentrations of P407 (0.6 g, equivalent to 6% w/w of the final sol-gel formulation) were added to a required amount of cold Milli-Q water (˜2-3 mL) and this solution was mixed thoroughly (400 rpm) for 45 min, at 2-8° C.
Optimised concentrations of HPMC (20 mg, equivalent to 0.2% w/w of the final sol-gel formulation), chitosan solution (10 mg, equivalent to 0.1% w/w of the final sol-gel formulation), glycerin as humectant/soothing agent (0.3 g, equivalent to 3% w/w of the final sol-gel formulation) and propyl paraben (2 mg, equivalent to 0.02% w/w of the final sol-gel formulation), were added to the cold P407 solution which was mixed thoroughly (400 rpm) for 45-60 min and left to fully hydrate overnight (12-14 h) at 2-8° C.
Finally, the lyophilised drug-micelles as dry powder was slowly added to the fully hydrated polymer-excipient mixture, which was then adjusted to pH≈5 with 0.1 M HCl or 0.1 N NaOH, and made up to 10 g (≈10 mL) with Milli-Q water.
The final drug micelle-infused sol-gel formulations were stored at cold temperature (2-8° C.) until required for further use. The total concentration of P407 in the sol-gel was 18% w/w (12% w/w from Phase 1 and 6% w/w from Phase 2).

TABLE 4
Size and polydispersity index of drugs (vit E, vit D or
cyclosporine)-loaded micelles (reconstituted in water).

| Formulation | Size (nm) | PDI |
|---|---|---|
| Vitamin D | 14.82 | 0.197 |
| Vitamin E | 15.21 | 0.278 |
| Cyclosporine-A | 16.08 | 0.306 |

TABLE 5
Composition of vitamin D, vitamin E and
cyclosporine-loaded sol-gel formulations.

| | P407 | HPMC | Chitosan |
|---|---|---|---|
| Formulation | (% w/w) | (% w/w) | (% w/w) |
| Vitamin D | 18 | 0.2 | 0.1 |
| Vitamin E | 18 | 0.2 | 0.1 |
| Cyclosporine-A | 18 | 0.2 | 0.1 |

All the formulations contain 3% w/w glycerin and 0.02% w/w propyl paraben.

TABLE 6
NTU value, gelation temperature, viscosity and gel strength of
vitamin D, vitamin E and cyclosporine-loaded sol-gel formulations.

| | | Gelation | Viscosity | Gel strength |
|---|---|---|---|---|
| | NTU | temperature | (Pa · s) | (Pa) |
| Formulation | value | (° C.) | (30° C.) | (30° C.) |
| Vitamin D | 6.92 | 21.4 | 0.768 | 12823.1 |
| Vitamin E | 8.34 | 20.8 | 0.704 | 14855.8 |
| Cyclosporine-A | 9.61 | 22.3 | 0.997 | 11065.4 |

### SUMMARY

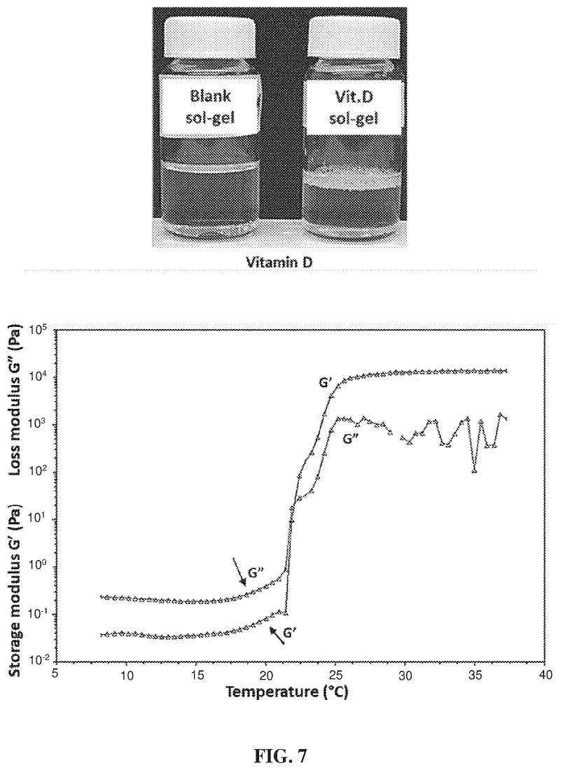

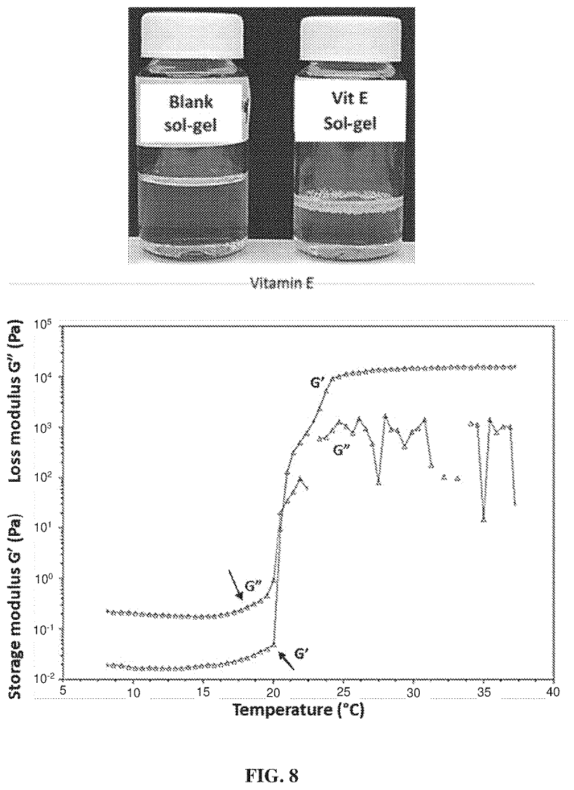

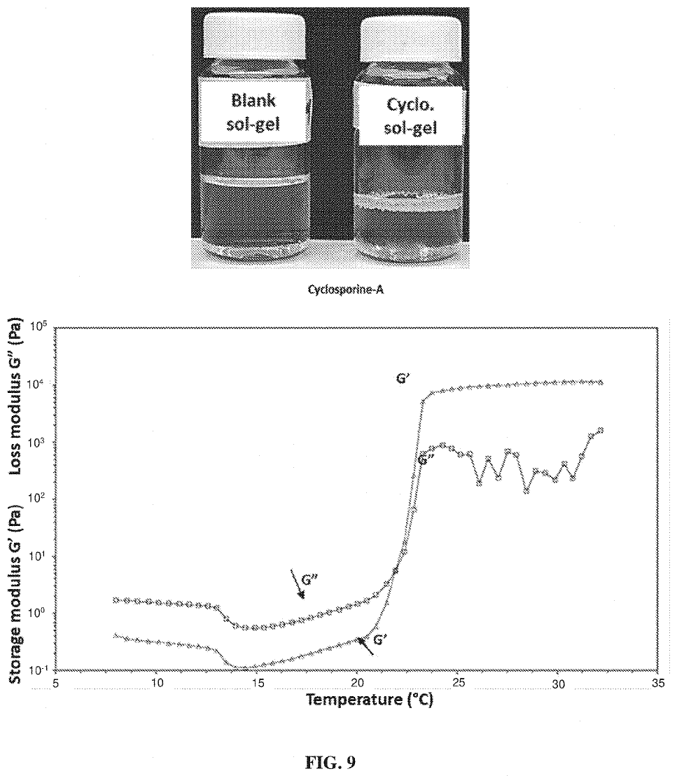

Three sol-gel formulations each infused with 0.1% w/w (1 mg/mL) of active (vit D, vit E or cyclosporine, respectively) were prepared using the aforementioned method, and were substantially free of particulates or suspended matter particulates (as assessed visually—see FIGS. 7-9, and confirmed by turbidimetry measurements) and with consistent micelle size and gelation temperatures attained. A higher gelation temperature can be achieved through decreasing the P407 concentration to circa. 15-16% w/w, which would need to be tailored for each active.

### Example 4

In this example, the long-term stability of a number of sol-gel compositions of the invention were tested.

TABLE 7
Sol-gel compositions of the invention

| | | | Visually | Turbidimetry |
|---|---|---|---|---|
| API/botanical | Storage | Time from | free of | measurement |
| (surfactant)¹ | temp | synthesis | particulates | (NTU) |
| MF (T80-5%)-1 | RT | 73 days | Yes | 23.64 |
| MF (T80-8%)-1 | RT | 73 days | Yes | 24.85 |
| MF (T80-5%)-2 | CT | 73 days | No | 109.4 |
| MF (T80-8%)-2 | CT | 73 days | No | 113.8 |
| MF (T80-5%)-3* | CT | 73 days | No | 34.24 |
| MF (T80-8%)-3* | CT | 73 days | No | 31.71 |
| CC (T80-2%) | RT | 90 days | Yes | 4.31 |
| CC (T80-5%)-1 | RT | 90 days | Yes | 2.03 |
| CC (T80-5%)-2 | RT | 90 days | Yes | 2.32 |
| KP (T20-5%) | RT | 80 days | Yes | 9.25 |
| KP (T20-8%) | RT | 80 days | Yes | 7.73 |
| KP (T40-5%) | RT | 80 days | Yes | 9.64 |
| KP (T40-8%) | RT | 80 days | Yes | 7.86 |
| KP (T80-5%) | RT | 81 days | Yes | 6.45 |
| KP (T80-8%) | RT | 81 days | Yes | 9.21 |
| DT (T20-2%) | RT | 75 days | Yes | 5.32 |
| DT (T20-5%) | RT | 75 days | Yes | 4.73 |
| DT (T20-8%) | RT | 75 days | Yes | 5.27 |
| DT (T40-2%) | RT | 74 days | Yes | 6.59 |
| DT (T40-5%) | RT | 74 days | Yes | 3.09 |
| DT (T40-8%) | RT | 74 days | Yes | 10.11 |
| DT (T80-2%) | RT | 73 days | Yes | 9.93 |
| DT (T80-5%) | RT | 73 days | Yes | 5.58 |
| DT (T80-8%) | RT | 73 days | Yes | 6.72 |
| PE (T20-2%) | RT | 70 days | Yes | 6.24 |
| PE (T20-5%) | RT | 70 days | Yes | 7.32 |
| PE (T20-8%) | RT | 70 days | Yes | 5.48 |
| PE (T40-2%) | RT | 71 days | Yes | 6.98 |
| PE (T40-5%) | RT | 71 days | Yes | 11.43 |
| PE (T40-8%) | RT | 72 days | Yes | 10.21 |
| PE (T80-5%) | RT | 72 days | Yes | 7.83 |
| PE (T80-8%) | RT | 72 days | Yes | 10.53 |

T80 = Tween ® 80,
T20 = Tween ® 20,
T40 = Tween ® 40,
RT = Room temperature (22-23° C.),
CT = cold temperature/fridge (2-8° C.),
MF = mometasone furoate,
CC = curcumin,
KP = ketoprofen,
DT = docetaxel,
PE = plant extract/botanical (Indian gooseberry extract (Emblica officinalis)),
HPMC = hydroxylpropyl methylcellulose,
SE = solvent evaporation.
¹The % of the surfactant given is the % w/w of the surfactant used based on the weight of the final sol-gel formulation.
*= after keeping the CT formulation at room temperature for 1 hr and then gentle agitation before testing
Note:
all formulations were prepared using Example 2 Method 4 described above (i.e. solvent evaporation). Formulations from the inventory, which gelled at room temperature or with microbial contamination/growth were excluded.

Key points:
Formulations MF (T80-5%)-1 & MF (T80-8%)-1 showing NTU values >10, were mostly free from visible particulates but not as clear or transparent, likely due to clumping of HPMC in the sol-gel system (disperses upon warming and gentle agitation). Stability of the drug in the system would need to be confirmed (RP-HPLC) to ascertain whether drug is precipitating, and if so, to what extent after 75 days of storage.
Formulations MF (T80-5%)-2 & MF (T80-8%)-2, showing NTU values >>10 immediately out from fridge, were turbid and thickened at CT. Plausible reason for turbidity was glycerol (melting point 17.8° C.).
When formulations MF (T80-5%)-2 & MF (T80-8%)-2 were kept at room temperature for about 1 hr and after a gentle agitation to avoid any bubble or foam, formulations MF (T80-5%)-3* & MF (T80-8%)-3* showed much lower NTU values 34.24 & 31.71, than previous ones i.e. 109.4 & 113.8, and their visually clarity improved with time. The likely reason being melting and redispersion of glycerol in the system.

### Example 5

### Synthesis

Sol-gel formulations infused with the hydrophobic therapeutic agents mometasome furoate (MF), curcumin (CC), ketoprofen (KP), docetaxel (DT) or an ethanolic extract of the Indian Gooseberry (Emblica officinalis) (PE) were prepared using a process consistent with Example 2 (Method 4). Note that these formulations were prepared with the following components and concentrations:

### Phase 1:

1.2 g of P407 (equivalent to 12% w/w of the final sol-gel formulation)
Various surfactants (Tween® 20, 40, 60, 80 or Solutol) and various surfactant amounts (0.2, 0.5, 0.8 g, equivalent to 2, 5 8% w/w of the final sol-gel formulation)
10 mg of the hydrophobic therapeutic agent (equivalent to 0.1% w/w of the final sol-gel formulation)

### Phase 2:

0.5 g of P407 (equivalent to 5% w/w of the final sol-gel formulation)
40 mg of HMPC (equivalent to 0.4% w/w of the final sol-gel formulation)
Thus, the final sol-gel formulation contains a total of 17% w/w of P407.
The table below shows that these prepared formulations possessed desirable turbidimetry values and physiologically acceptable gelation temperatures. A higher or lower gelation temperature can feasibly be tailored for each therapeutic agent by decreasing or increasing the P407 concentration respectively.

TABLE 8
Details of sol-gel compositions

| | | | Gelation |
|---|---|---|---|
| Hydrophobic | | | temperature |
| Agent | Surfactant¹ | NTU values | (° C.) |
| MF | T80 (5%) | 1.2 | 27.38 |
| MF | T80 (8%) | 0.75 | 33.54 |
| CC | T80 (2%) | 2.13 | 26.57 |
| CC | T80 (5%) | 0.512 | 28.30 |
| CC | Solutol (2%) | 5.61 | 25.15 |
| CC | Solutol (5%) | 10.1 | 27.48 |
| CC | Solutol (8%) | 16.3 | 34.04 |
| KP | T20 (8%) | 5.16 | 33.5 |
| KP | T40 (5%) | 8.12 | 28.2 |
| DT | T20 (2%) | 4.96 | 24.3 |
| DT | T20 (5%) | 2.96 | 34.4 |
| DT | T20 (8%) | 4.82 | 35.2 |
| DT | T40 (2%) | 5.13 | 24.5 |
| DT | T80 (2%) | 8.12 | 29.5 |
| DT | Solutol (2%) | 8.24 | 26.3 |
| PE | T20 (2%) | 4.82 | 25.18 |
| PE | T40 (2%) | 5.89 | 23.71 |
| PE | T40 (5%) | 9.82 | 32.54 |

¹The % of the surfactant given is the % w/w of the surfactant used based on the weight of the final sol-gel formulation.

### Example 6

The present example describes embodiments of a gel-based formulation that are loaded with micelles containing a hydrophobic therapeutic agent (see FIG. 11).
Poloxamer (P407) based gel

### Method:

Step 1: Preparation of ketoprofen (KP)-loaded polymeric micelles:
KP-loaded polymeric micelles were first prepared by the solvent evaporation method i.e. Example 2, Method 4:
10 mg of KP was added to 10 mL of acetone forming a clear drug solution (equivalent to 0.1% w/w KP of the final gel formulation).
The aforementioned drug solution was mixed with 1.2 g of P407 (equivalent to 12% w/w of the final gel formulation) and 0.8 g of Tween® 80 (equivalent to 8% w/w of the final gel formulation) in 8 mL deionized water.
The mixture was stirred thoroughly (400 rpm) for 30 minutes at room temperature.
Acetone was completely removed by rotary evaporation (34° C. water bath, for 1 h) to obtain a dual-polymeric KP-micellar solution.
The resultant solution was then filtered (0.22 μm) to remove traces of unentrapped/undissolved KP.
The eluted KP-micellar solution was lyophilized, and stored at 2-8° C. until required.
   Step 2: Preparation of a single phase, poloxamer gel system containing KP-micelles:
0.8 g of P407 (equivalent to 8% w/w of the final gel formulation) was added to cold deionized water (4-5 mL) and mixed thoroughly (400 rpm) for 45 min at 2-8° C.
The lyophilized KP-micelle powder (equivalent to 0.1% w/w KP) was added slowly to this poloxamer preparation with continuous stirring (300 rpm) and mixed properly at room temperature.
This micelles-containing poloxamer gel was made up to 10 g (˜10 mL) with deionized water and stirred again properly at room temperature and stored at room temperature until required.
   Carbomer (carbopol 934 P) based gel

### Method:

Step 1: Preparation of ketoprofen (KP)-loaded polymeric micelles:
KP-loaded polymeric micelles were first prepared by the solvent evaporation method i.e. Example 2, Method 4:
10 mg of KP was added to 10 mL of acetone forming a clear drug solution (equivalent to 0.1% w/w KP of the final gel formulation).
The aforementioned drug solution was mixed with 1.2 g of P407 (equivalent to 12% w/w of the final gel formulation) and 0.8 g of Tween® 80 (equivalent to 8% w/w of the final gel formulation) in 8 mL deionized water.
The mixture was stirred thoroughly (400 rpm) for 30 minutes at room temperature.
acetone was completely removed by rotary evaporation (34° C. water bath, for 1 h) to obtain a dual-polymeric KP-micellar solution.
the resultant solution was then filtered (0.22 μm) to remove traces of unentrapped/undissolved KP.
the eluted KP-micellar solution was lyophilized, and stored at 2-8° C. until required.
   Step 2: Preparation of a single phase, carbomer gel system containing KP-micelles:
0.05 g of carbopol 934 P (equivalent to 0.5% w/w of the final gel formulation) was dispersed gently into 6-7 mL water with constant stirring using magnetic stirrer so that no lump remains in the dispersion.
To obtain carbopol gel, the pH of carbopol dispersion was adjusted to pH 5-6 using required amount of triethanolamine.
Then lyophilized KP-micelle powder (equivalent to 0.1% w/w KP) was added slowly to this carbomer dispersion with continuous stirring (300 rpm) and mixed properly at room temperature.
This micelle-containing polymeric gel weight was made up to 10 g (˜10 mL) with deionized water, stirred properly and stored at room temperature.

1. A method of preparing a thermo-responsive sol-gel composition, including the steps of:
(a) providing a mixture of a first aqueous solution comprising a first poloxamer and/or a first poloxamine with a solvent solution comprising a water miscible solvent and a hydrophobic therapeutic agent, wherein the water miscible solvent has a boiling point of less than 105° C. at atmospheric pressure and wherein the first aqueous solution and/or the solvent solution further comprise a surfactant;
(b) substantially removing the water miscible solvent and water from the mixture in (a) to produce a micelle composition;
(c) contacting the micelle composition with a second aqueous solution comprising a second poloxamer and/or a second poloxamine to thereby prepare the thermo-responsive sol-gel composition.

2. A method of preparing a gel-based composition, including the steps of:
(a) providing a mixture of a first aqueous solution comprising a first poloxamer and/or a first poloxamine with a solvent solution comprising a water miscible solvent and a hydrophobic therapeutic agent, wherein the water miscible solvent has a boiling point of less than 105° C. at atmospheric pressure and wherein the first aqueous solution and/or the solvent solution further comprise a surfactant;
(b) substantially removing the water miscible solvent and water from the mixture in (a) to produce a micelle composition;
(c) contacting the micelle composition with a second aqueous solution comprising a second poloxamer, a further polymer and/or a second poloxamine to thereby prepare the gel-based composition.

3. A method of preparing a micelle composition, including the steps of:
(a) providing a mixture of a first aqueous solution comprising a first poloxamer and/or a first poloxamine with a solvent solution comprising a water miscible solvent and a hydrophobic therapeutic agent, wherein the water miscible solvent has a boiling point of less than 105° C. at atmospheric pressure and wherein the first aqueous solution and/or the solvent solution further comprise a surfactant;
(b) substantially removing the water miscible solvent and water from the mixture in (a) to thereby produce the micelle composition.

4. The method of claim 1, further including the steps of:
(a) mixing the first aqueous solution and the solvent solution; and/or
(b) preparing the first aqueous solvent, the second aqueous solvent and/or the solvent solution.

5. The method of claim 1, wherein the first aqueous solution comprises the surfactant.

6. The method of claim 1, wherein the water miscible solvent is or comprises a ketone and/or a primary alcohol.

7. The method of claim 6, wherein the water miscible solvent is selected from the group consisting of acetone, methanol, ethanol, propanol, butanol, pentanol, hexanol and any combination thereof.

8. The method of claim 1, wherein the first and/or the second poloxamer are or comprise P407 (F127).

9. The method of claim 1, wherein the step of removing the water is performed at least in part by lyophilization.

10. The method of claim 1, wherein the step of removing the water miscible solvent is performed at least in part by rotary evaporation.

11. The method of claim 1, wherein the step of removing the water miscible solvent is performed at a temperature from about 25° C. to about 35° C.

12. The method of claim 11, wherein the step of removing the water miscible solvent is performed at a temperature from about 32° C. to about 34° C.

13. A thermo-responsive sol-gel composition or a gel-based composition for therapeutic use comprising:
an aqueous solution;
a carrier micelle disposed within the aqueous solution and including a poloxamer and/or a poloxamine and a surfactant and having a hydrophobic core; and
a hydrophobic therapeutic agent disposed within the hydrophobic core of said carrier micelle.

14. The thermo-responsive sol-gel composition, or gel-based composition of claim 13, wherein the carrier micelle is about 10 nm to about 25 nm in diameter.

15. The thermo-responsive sol-gel composition, or gel-based composition of claim 13, wherein the poloxamer is or comprises P407.

16. The method of claim 1 or the thermo-responsive sol-gel composition or gel-based composition, wherein the thermo-responsive sol-gel composition has a gelation temperature below about 35° C.

17. The method, thermo-responsive sol-gel composition or gel-based composition of claim 16, wherein the gelation temperature is between about 20° C. to about 32° C.

18. The method, thermo-responsive sol-gel composition or gel-based composition of claim 1, wherein the thermo-responsive sol-gel composition has a viscosity of less than about 0.15 Pa·s at about 22° C. and greater than about 0.3 Pa·s at about 30° C. or at about 35° C.

19. The method, thermo-responsive sol-gel composition or gel-based composition of claim 1, wherein the thermo-responsive sol-gel composition has a gel strength of greater than about 500 Pa at about 30° C. or about 35° C.

20. The method, thermo-responsive sol-gel composition, or gel-based composition of claim 1, wherein the surfactant is selected from the group consisting of a polyoxyethylated sorbitan fatty ester, a polyoxyethylated glycol monoether, a polyoxyethylated glyceride, n-dodecyl tetra (ethylene oxide), a polyoxyethylated fatty acid, a polyoxyethylated castor oil, a sucrose ester, a lauroyl macroglyceride, a polyglycolyzed glyceride and any combination thereof.

21. The method, thermo-responsive sol-gel composition, or gel-based composition of claim 20, wherein the surfactant is or comprises polyoxyethylene sorbitan monooleate.

22. The method, thermo-responsive sol-gel composition, or gel-based composition of claim 1, wherein the hydrophobic therapeutic agent is selected from the group consisting of a steroid, an anticancer agent, an antifungal agent, an anti-inflammatory agent, a sex hormone, an immunosuppressant, an antiviral agent, an antibacterial agent, an anti-fibrotic agent, an antihistamine agent, a vitamin, a plant extract and any combination thereof.

23. A method of administering a hydrophobic therapeutic agent to a subject, the method including the step of administering the thermo-responsive sol-gel composition or the gel-based composition of claim 13 to the subject.

24. A method of preventing and/or treating a disease, disorder or condition in a subject, including the step of administering to the subject a therapeutically effective amount of the thermo-responsive sol-gel composition or the gel-based composition of claim 13 to thereby prevent and/or treat the disease, disorder or condition.

25. The method of claim 24, wherein the disease, disorder or condition is or comprises a respiratory disease, disorder or condition.
